(19) United States
(12) Patent Application Publication
(10) Pub. No.: US 2025/0303145 A1
(43) Pub. Date: Oct. 2, 2025

(54) CUFF ELECTRODE FOR PHRENIC NERVE STIMULATION

(71) Applicant: LUNAIR MEDICAL, Minneapolis, MN (US)

(72) Inventors: Eric Daniel BUCK, Shoreview, MN (US); Avram SCHEINER, Vadnais Heights, MN (US); Edward Frederick RUPPEL, III, Saratoga, CA (US); Orhan SOYKAN, Lino Lakes, MN (US)

(21) Appl. No.: 19/091,420

(22) Filed: Mar. 26, 2025

Related U.S. Application Data
(60) Provisional application No. 63/570,459, filed on Mar. 27, 2024.

(51) Int. Cl. A61N 1/05; A61N 1/08; A61N 1/36

(52) U.S. Cl. CPC A61N 1/0556; A61N 1/0558; A61N 1/08; A61N 1/3611

(57) ABSTRACT

A two-piece electrode cuff to stimulate a nerve is provided. The cuff is arranged to adapt it to the natural form of the nerve as well as to the surrounding tissues. The shape of the electrode cuff is optimized for ease of placement and it assures that the device will remain in place without major displacement and will minimize the damage to the nerve post-implant.

### CROSS-REFERENCE TO RELATED APPLICATIONS

This application claims priority to U.S. Provisional Application No. 63/570,459, filed Mar. 27, 2024, the entire contents of which are hereby incorporated by reference.

### TECHNICAL FIELD

The devices described herein relate to the method of making electrical connections to the phrenic nerve using implantable devices that are designed to treat sleep-disordered breathing-such as airway collapse in patients with Obstructive Sleep Apnea (OSA). The devices are designed to be easily implanted with minimal injury to the surrounding tissues. Features of the device that reduce the chances of device displacement and nerve damage are also disclosed.

### BACKGROUND

### Sleep Apnea:

Healthy sleep is an important part of our lives. It improves physical and mental health. Sleep happens in stages, including REM sleep and non-REM sleep. When humans sleep, their body has a chance to rest and restore energy. A good night's sleep can help us cope with stress, solve problems, or recover from illness. Not getting enough sleep can lead to health concerns, and can affect how we think and feel.

During sleep, a person usually passes through four sleep stages: non-REM N1, N2, and N3, and REM (rapid eye movement). These stages of sleep progress in a cycle from N1 to REM sleep, then the cycle starts over again with N1 or N2. Healthy children and adults spend almost 50 percent of their total sleep time in N2 sleep, about 20 percent in REM sleep, and the remaining 30 percent in the other stages.

During N1, which is light sleep, we drift in and out of sleep and can be woken easily. Our eyes move very slowly, and muscle activity slows. People that wake from N1 sleep often remember fragmented visual images.

When we enter N2 sleep, our eye movements stop and our brain waves (fluctuations of electrical activity that are measurable by an electroencephalogram (EEG) become slower, with occasional bursts of rapid waves called sleep spindles.

A sleep EEG is a recording of the electrical activity of the brain while a person is awake and then asleep. It involves having small electrodes that record the brain activity attached to the scalp.

In N3, delta waves (extremely slow brain waves) begin to appear, interspersed with smaller, faster waves until delta waves occur almost exclusively. It is usually more difficult to wake someone during N3, which is also called deep or slow wave sleep.

Once we enter REM sleep, our breathing becomes more rapid, irregular, and shallow, our eyes jerk rapidly in various directions, and our limb muscles become temporarily paralyzed during sleep. Our heart rate increases, and our blood pressure rises. When people wake up during REM sleep, they often describe dreams.

The first REM sleep period usually occurs about 70 to 90 minutes after we fall asleep. A complete sleep cycle takes 90 to 110 minutes on average. The first sleep cycles each night contain relatively short REM periods and long periods of deep sleep. As the night progresses, REM sleep periods increase in length while deep sleep decreases. By morning, healthy people spend nearly all their sleep time in stages 1, 2, and REM.

Although the neurophysiology of sleep is not completely understood, it is indisputable that a good night of sleep is a night of continuous, uninterrupted sleep that cycles through sleep stages, including REM. Sleep disorders, specifically those under the category of sleep apnea syndrome, frequently interrupt these continuous sleep patterns and lead to daytime sleepiness, fatigue, and have many other serious deleterious effects on both mental and physical health.

Sleep-disordered breathing is a common sleep disorder where patients have repetitive episodes of either cessation of breathing (apneas) or periods of reduced flow (hypopneas) during sleep. For patients with sleep-disordered breathing, sleep is interrupted with 10 or more second periods without proper airflow, occurring hundreds of times during a typical night's sleep. Apneas generally originate as either obstructive, central, or some combination of the two etiologies.

Obstructive Sleep Apnea (OSA) is a well-recognized disease that affects millions of people. It is a form of sleep-disordered breathing characterized by periodic interruptions of lung ventilation that disrupt sleep due to a momentary collapse and obstruction of the pharyngeal airway. Obstruction of the pharyngeal airway can be attributed to decreased upper airway muscle tone, relaxing muscles that support the soft tissues in the throat, such as the tongue and/or soft palate. Relaxation of these muscles results in the narrowing of the pharyngeal airway, causing airflow obstruction thus limiting airflow and leading to a decrease in oxygen saturation. Central Sleep Apnea (CSA) is a less common sleep disorder that is characterized by apneas due to a lack of signals from the respiratory center. With CSA, thoracic neural receptors fail to send a signal to the respiratory center to initiate inspiration. As a result, airflow ceases due to no respiratory muscle activity. Mixed Sleep Apnea is a combination of OSA and CSA where there is both decreased respiratory drive and decreased upper airway muscle tone.

Pathogenesis of the Upper Airway (UA) obstruction during sleep is due to (a) a primary sleep-related loss of UA neuromotor tone and (b) a lack of adequate compensatory reflex responses that mitigate the obstruction. The inventors believe that OSA may be caused by an inadequate reflex mechanism in response to an obstructed airway.

### Control of Respiration:

In healthy individuals, upper airway stability during sleep is ensured by coordinated and synchronized central control of the respiratory system, specifically the airway muscles, which are comprised of about twenty airway dilator and constrictor muscles. The central nervous system (CNS) pattern generator, also referred to as the respiratory control system, in the medulla of the brain receives inputs from physiologic sensors (also called receptors) via various afferent sensory nerve fibers and controls airway muscles via efferent motor fibers. These physiologic sensors provide physiologic feedback used by the medulla to trigger a reflex from the effectors in a closed-loop reflex arrangement. These reflexes are known as “autonomic” since they do not depend on consciousness. In some cases, the reflexes become insufficient for optimal health during sleep.

Respiration during sleep is governed mainly by three systems as illustrated in FIG. 1, namely the Central Neural Controller, the respiratory system itself, and the cardiovascular system. The brainstem, cortex, limbic system, and hypothalamus primarily contribute to respiratory effort from the central neural controller. The central pattern generator from the brainstem controls the periodic nature of inspiration and expiration. Three main groups of neurons located in the pons and medulla aid in generating rhythmic breathing: the medullary respiratory center, apneustic center, and pneumotaxic center. The medulla respiratory center is comprised of different groups of cells that are responsible for the basic rhythm of ventilation, including the generation of respiratory rhythm, inspiration, and expiration. These cells generate repetitive bursts of action potentials without afferent stimuli to send nervous impulses to the diaphragm and other inspiratory muscles. The rhythmic pattern of inspiration begins with an initialization of several seconds where no activity occurs. Action potentials then occur to create a crescendo for a period of several seconds, causing inspiratory muscle activity to become stronger. The inspiratory action potentials then cease, and the inspiratory muscle tone falls to its preinspiratory level. The apneustic center creates impulses that have an excitatory effect on the inspiratory area of the medulla, prolonging the action potentials, causing abnormal breathing. The pneumotaxic center regulates inspiration volume and respiration rate by inhibiting inspiration. However, a normal breathing pattern can exist without the pneumotaxic center, leading scientists to believe that this center's role is for “fine-tuning” the respiratory rhythm.

The cortex may override the function of the brainstem in certain situations, such as hyperventilation or voluntary hypoventilation. Other parts of the brain, such as the limbic system and hypothalamic, can alter rhythmic breathing as well, due to different emotional states.

Sensory inputs to the respiratory center include signals from chemoreceptors and many distributed mechanoreceptors. Central chemoreceptors are involved with the minute-by-minute control of ventilation and react to the amount of CO₂ dissolved in the blood (PCO₂), but not the amount of oxygen (PO₂). In addition, central chemoreceptors respond to changes in hydrogen ion (H⁺) concentrations, where an increase in H⁺ concentration stimulates ventilation, and a decrease in H⁺ concentration slows respiration rate. Peripheral chemoreceptors respond to a decrease in arterial PO₂, an increase in PCO₂, a change in H⁺, and a change in arterial pH.

Afferent receptors in the tracheobronchial tree and lungs detect alterations in airway pressure, temperature, airflow, and lung stretch which may be indicators of a collapsed airway. The afferent receptors provide feedback signals to the spinal cord or CNS which may respond to the feedback signals by triggering reflex responses that stimulate the upper airway muscles, which can then mitigate airway obstruction.

Some of the afferent receptors that aid in ventilation and respiration are mechanoreceptors. Lung receptors are one type of afferent receptor that provide inputs carried via the vagus nerve to the CNS to influence ventilation. Pulmonary stretch receptors are a type of lung receptor located in the smooth muscle of the airway walls that respond to changes in lung inflation. That is, these stretch receptors contribute to switching off inspiration and initiate exhalation based on how inflated the lungs are. Feedback from these stretch receptors inhibits further inspiratory muscle activity as the lungs inflate, and initiation of inspiratory activity results in a deflation of the lungs.

Other types of lung receptors include irritant receptors, J-receptors, and bronchial C fibers. Irritant receptors are stimulated by inhaled noxious stimuli such as cigarette smoke or inhaled dust. These receptors are more rapidly adapting than stretch receptors and result in tachypnea. J-receptors also cause tachypnea, dyspnea, and apnea, as a result of events such as pulmonary edema, pulmonary emboli, pneumonia, etc. Bronchial C fibers are supplied by bronchial circulation rather than pulmonary circulation and respond to chemicals injected into bronchial circulation. Stimulation of the bronchial C fibers also results in tachypnea. FIG. 2 shows the various sensors and their afferent nerves that carry the information regarding respiration and ventilation to the central nervous system (CNS). Afferent and efferent pathways with CNS involvement are shown as a block diagram in FIG. 3.

Additional receptors also can impact ventilation and respiration. These include receptors in the nose, nasopharynx, larynx, and trachea. These receptors respond to, for example, mechanical and chemical stimulation—e.g., irritants. Joint and muscle receptors impact ventilation by sending signals during exercise. Receptors in the intercostal muscles and diaphragm contain muscle spindles that sense elongation of the muscle. These receptors adjust the output of respiratory muscles if the degree of muscular work has not been met or has been exceeded, helping to control the strength and degree of contraction. When unusually large respiratory efforts are required to move the lung and chest wall, dyspnea occurs due to the discrepancy between the output from the CNS controller and the amount of stretch sensed by these receptors. Arterial baroreceptors can cause reflex hypoventilation or apnea through stimulation of the aortic and carotid sinus baroreceptors. Accordingly, many afferent nerves can induce changes in ventilation.

### Sensory (Afferent) Nerves:

Sensory nerves, also known as afferents, carry information from the peripheral organs to the central nervous system. They respond to the sensory stimulation by changing their firing rate, as illustrated in FIG. 4. In the example that is shown, the firing frequency of the sensory nerve begins to increase, in this case from zero, as the input stimulus is increased. The nerve firing rate saturates at f_{MAX} once the stimulus level exceeds PMAX. It should be noted that some sensory nerves maintain a non-zero firing rate even in the absence of any physical stimuli.

In addition to having a non-linear input-output relationship as shown in FIG. 4, sensor nerves also have a time-dependent response to the physical stimulus that they receive. Although they produce a rapid response to the initial stimulus, their response to the same level of stimulus does decrease over time. This phenomenon is further illustrated in FIG. 5 with the use of a model of the sensory nerve.

As shown in FIG. 5, the output of the sensory nerve is its firing frequency, f_{OUT}, which is determined as a sum of two signals at [0550]. The first signal contributing to four comes from the lower pathway that is shown in FIG. 5 and is the product of input stimulus, P_{IN}, and K_{P} [0540], K_{P} where represents a proportionality constant. The second signal contributing to four is produced by the upper pathway shown in FIG. 5 and is a result of the changes in the input stimulus P_{IN}. K_{D} represents a proportionality constant for the rate of change-related signal contributing to four. The remaining two items in the upper pathway are the rate of change determining differentiator [0520] and positive-only determinant [0530]. The last item that is mentioned, the positive-only determinant, indicates that the rate of change response that is described earlier applies only to the onset of the physical stimulation, and is absent at the conclusion of the physical stimulation.

### Motor (Efferent) Nerves:

Motor nerves, also known as efferents, carry information from the central nervous system to peripheral organs. They provide the excitation to the muscles, such as the diaphragm of the respiratory system. A typical firing pattern of a motor neuron is shown in FIG. 6. A normal firing duration of the motor neuron is usually 300 milli-seconds, as shown by the train of firings from t=100 milli-seconds to t=400 milli-seconds in FIG. 6. It should be noted that the firing frequency of the motor neuron is not fixed, but it increases as a function of time. In the example that is shown in FIG. 4, the firing frequency increases from 40 Hertz to 60 Hertz, which may or may not be linear. Most of the time, the recorded signal is too noisy to be analyzed in detail, hence its time integral is derived. Furthermore, a smoothed version of the time integral is used for signal processing purposes.

Since the motor neurons carry excitation signals to the nerves, they tend to have large cross-sections. However, the distribution of cross-sectional areas of motor axons in the phrenic nerve can change as a function of aging, as illustrated in FIG. 7. As the individual ages, the distribution shifts toward the motor neurons with smaller cross-sectional areas.

### Electrical Stimulation of Nerves:

As with other electrically excitable tissues, both the motor nerves and the sensory nerves can be stimulated with externally applied electrical signals. This can be done with various types of electrodes, such as the cuff electrode as shown in FIG. 8. Typical strength-duration curves for the capture of sensory and motor nerves are shown in FIG. 9. Due to the reduced cross-sectional area of the sensory nerves, larger pulse widths are required to capture them compared to the motor nerves. Following the delivery of the electrical stimulation to the nerve, a resulting action potential travels through the nerve fiber, as shown in FIG. 10. It should be noted that even though the duration of the action potential waveform that is shown in FIG. 10 is rather short, muscle contractions that last much longer can be achieved by repeated application of the stimulation train to generate a sustained tetanic contraction.

Waveforms that are used for the electrical stimulation of nerves are shown in FIG. 11, where FIG. 11A shows a monopolar stimulation waveform and FIG. 11B shows a bipolar stimulation waveform. The following list of parameters of the stimulation waveform is programmable:
Amplitude [1120a and 1120b]
Pulse-width [1130a, 1130b and 1130c]
Pulse period [1110a and 1110b]
Train duration [1140a and 1140b]
Silence duration [1145a and 1145b]

Furthermore, the stimulation can be delivered as a voltage or a current waveform, and may or may not be constant during the actual stimulation duration, i.e. pulse-width.

### SUMMARY OF THE INVENTION

The invention is directed to a medical device that can be used to make electrical connections to a phrenic nerve. Related systems and methods for the build of the device as well as the design parameters are provided.

A two-piece electrode cuff to stimulate a nerve is disclosed. The cuff has a special geometry to adapt it to the natural form of the nerve itself as well as to the surrounding tissues. The shape of the electrode cuff is optimized for ease of placement and it assures that the device will remain in place without major displacement and will minimize the damage to the nerve post-implant.

Furthermore, the design of the electrode cuff was done such that it minimizes the amount of energy that is required for the excitation of the nerve.

Additional features of the electrode cuff further allow it to work with different patients, different sensing and stimulation configurations, and with different modes of connection to the implantable device.

### BRIEF DESCRIPTION OF THE DRAWINGS

Some aspects of the present invention will be best understood when read in conjunction with the accompanying drawings, wherein:

FIG. 1 shows the three systems governing respiration during sleep,

FIG. 2 shows the various sensors and their afferent nerves that carry the information regarding respiration and ventilation to the central nervous system,

FIG. 3 shows the pathways involved with breathing control, including afferent and efferent pathways with CNS participation,

FIG. 4 shows the relationship between the firing frequency and drive stimulus for a neuron,

FIG. 5 shows a simplified block diagram of a sensory neuron,

FIG. 6 shows the various features of the waveform of a motor neuron while it is firing,

FIG. 7 shows how the composition of motor axons in the phrenic nerve changes with age,

FIG. 8 shows a tripolar cuff electrode for nerve stimulation,

FIG. 9 shows the strength-duration curves for motor and sensory neurons,

FIG. 10 shows the morphology of an electrically evoked compound action potential of a motor neuron,

FIGS. 11A and 11B shows unipolar and bipolar stimulation waveforms and their parameters, where FIG. 11A shows a monopolar stimulation waveform and FIG. 11B shows a bipolar stimulation waveform,

FIG. 12 shows the clinical configuration of the phrenic nerve monitoring and modulation system during its training phase,

FIGS. 13A to 13C show a schematic of a cuff electrode to stimulate the phrenic nerve, where FIG. 13A shows an isometric view of the electrode cuff, FIG. 13B shows a sectional view of the electrode cuff, and FIG. 13C shows an exploded view of the electrode cuff,

FIG. 14 shows an electrode cuff with a lead connector that minimizes torque on the cuff electrode,

FIG. 15 shows an additional embodiment of a lead connector that minimizes torque on the electrode cuff,

FIG. 16 shows an electrode cuff with a lead connector that prevents the lead from crossing itself,

FIG. 17 shows a unilateral cuff electrode to stimulate the phrenic nerve superiorly,

FIG. 18 shows a different embodiment of a unilateral cuff electrode to stimulate the phrenic nerve inferiorly,

FIG. 19A illustrates a monopolar cuff electrode for bidirectional stimulation,

FIG. 19B shows a multi-contact cuff electrode that is used for bipolar stimulation, and the biphasic stimulation waveforms, resulting in bi-directional excitation of the target nerve,

FIG. 19C shows the unidirectional nerve stimulation when a multi-contact cuff electrode is used as a nerve blocker,

FIG. 20 shows a stimulation waveform based on the respiratory cycles of a patient,

FIG. 21 shows a lead with multiple lumens for wiring through the lead,

FIG. 22 shows a concentric lead design with multiple conductors, FIG. 23 shows a multiple conductor spiral lead design,

FIG. 24 shows a lead connected to a cuff electrode where the shape of the lead is altered by way of a tension string,

FIGS. 25A to 25C shows fixation of an electrode to muscle by way of spring-loaded hooks. FIG. 25A illustrates a side view of an electrode cuff with spring-loaded hooks for mounting to muscle. FIG. 25B illustrates the cuff electrode before deployment out of a sleeve, that is, before the cuff electrode is mounted to muscle. FIG. 25C illustrates an electrode cuff mounted into the muscle by way of spring-loaded hooks,

FIG. 26 shows the placement of the contacts within the cuff electrode as well as the orientation of the Cartesian Coordinates with respect to the overall geometry of the cuff electrode,

FIG. 27 shows the values of the voltages over on a constant Y plane,

FIG. 28 shows the component of the electrical field along the Z axis on a constant Y plane,

FIG. 29 shows the component of the gradient of the electrical field along the Z axis on a constant Y plane,

FIG. 30 shows the X, Y, Z dimensions of the overall geometry of the cuff electrode, respective to the cuff electrode placement, and

FIG. 31 shows the dimensions of the cuff electrode in the X, Z Axis.

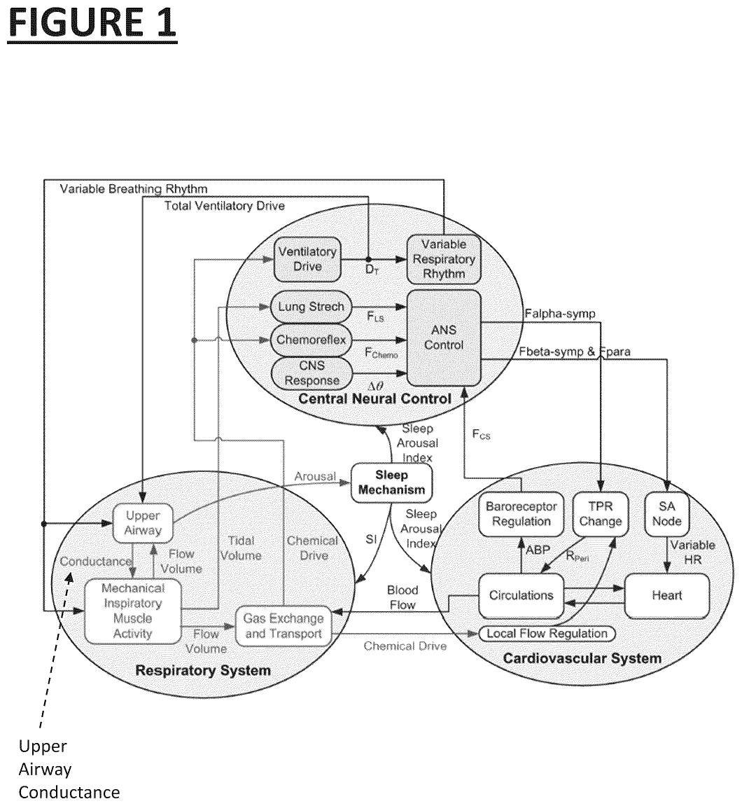

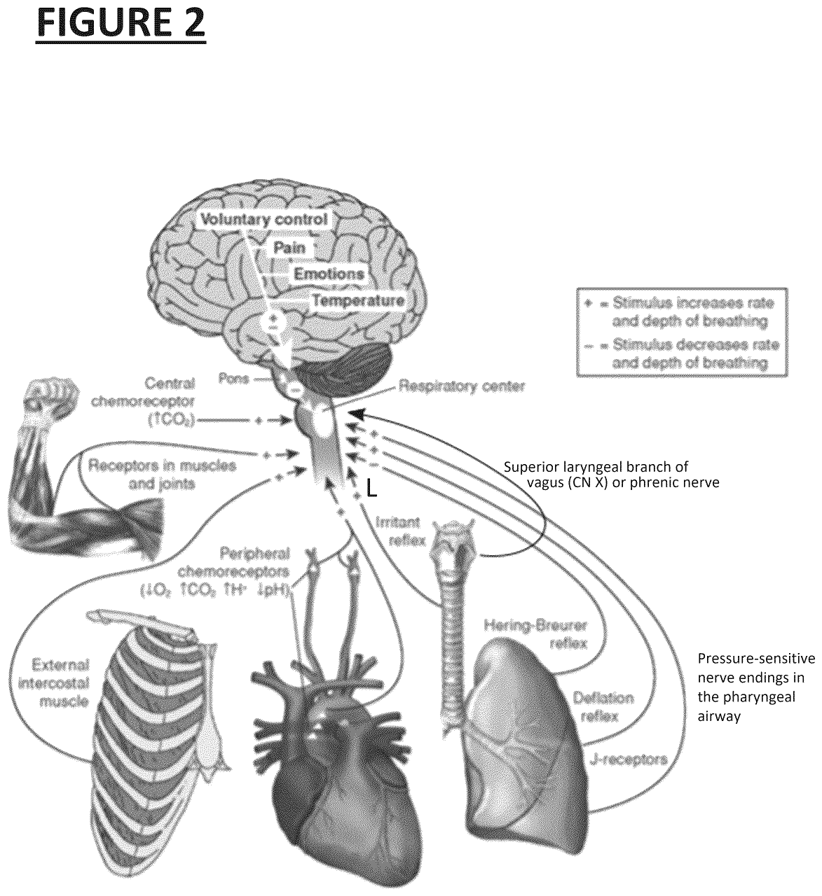

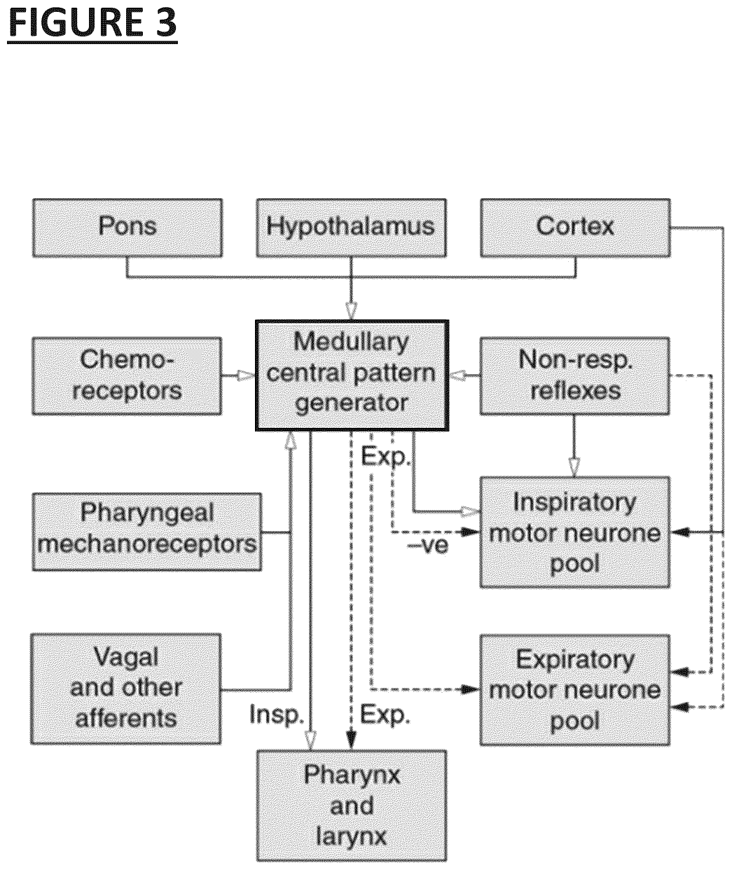

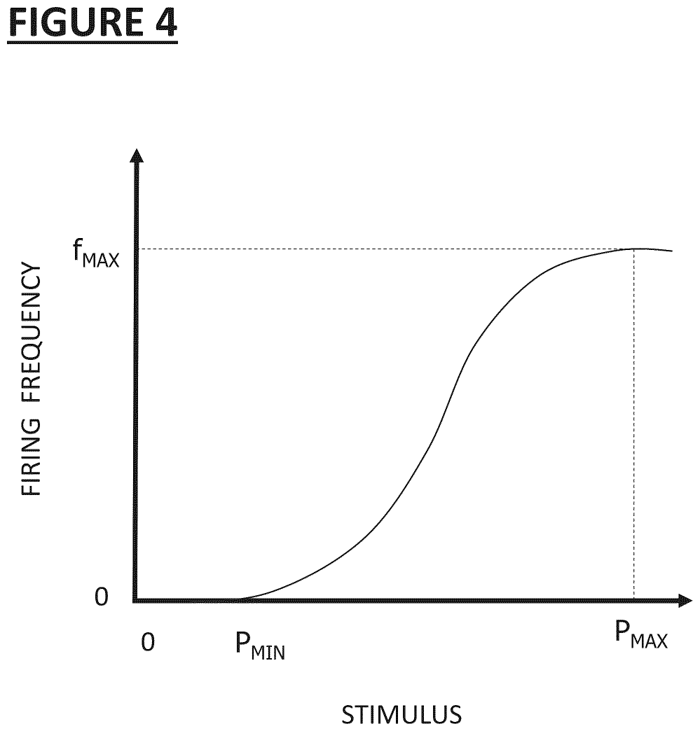

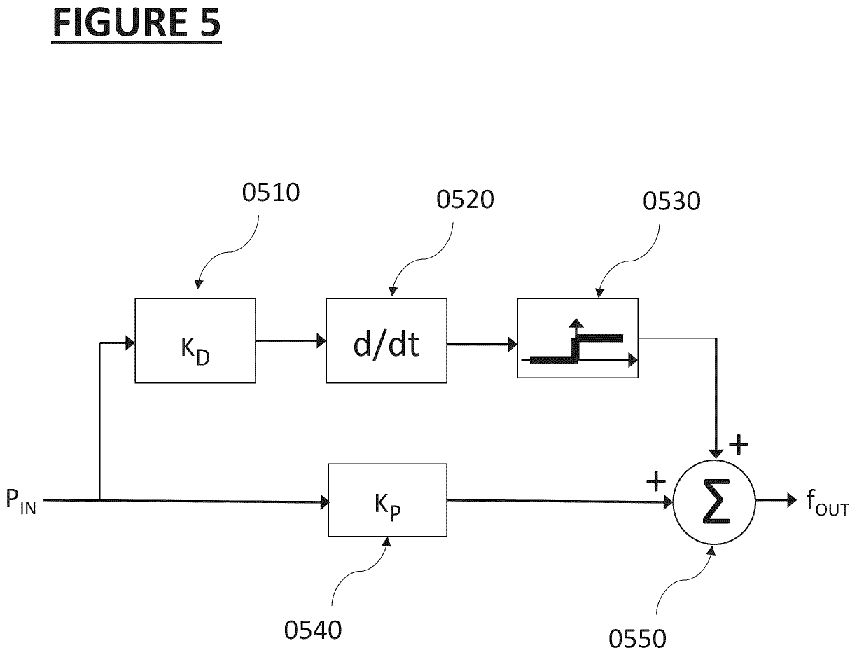

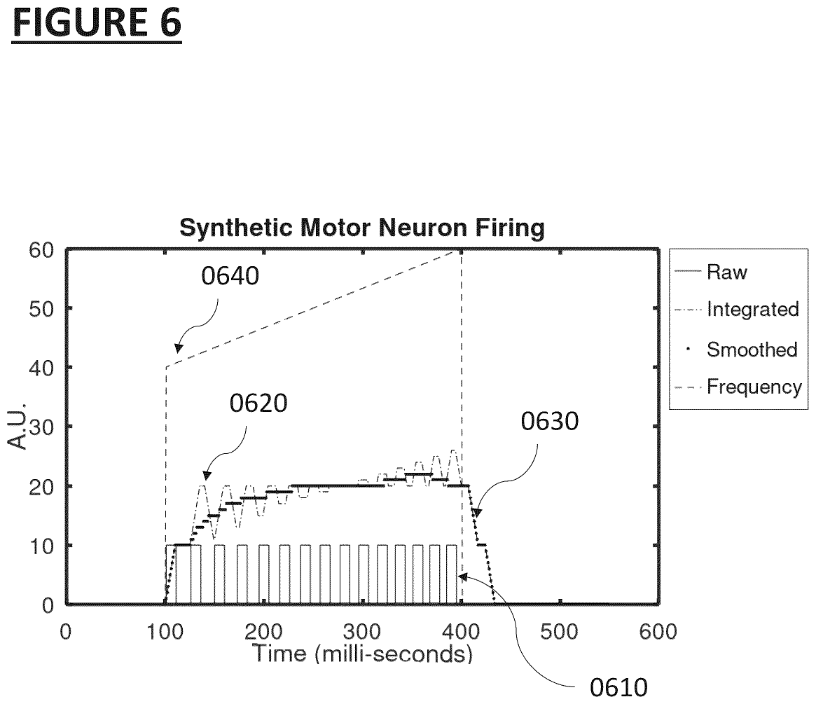

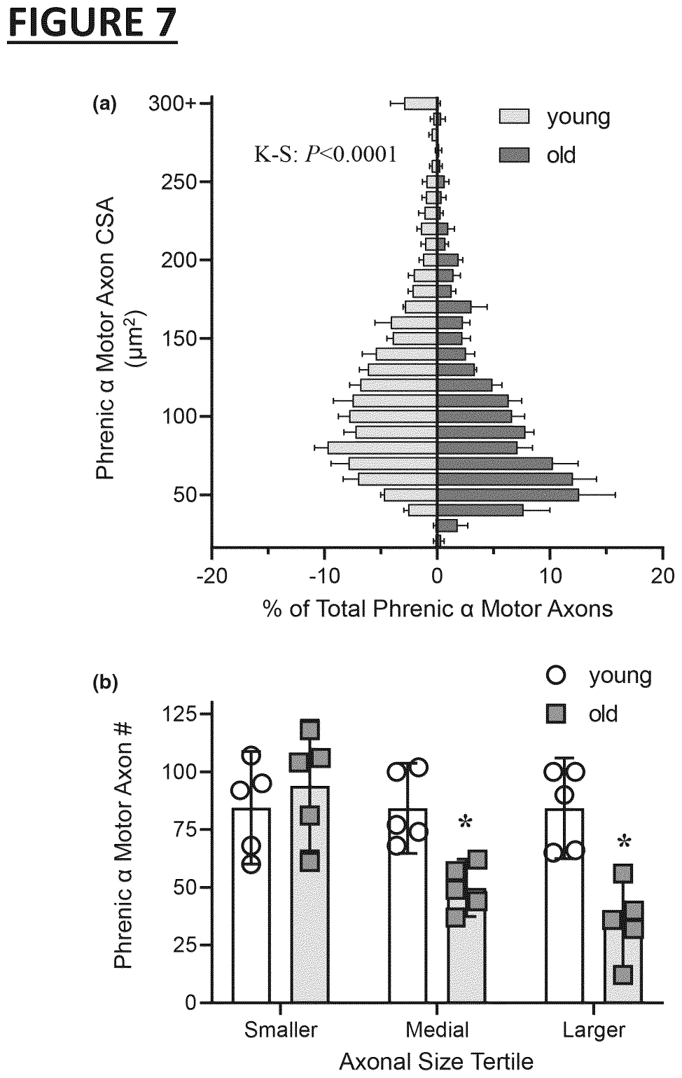

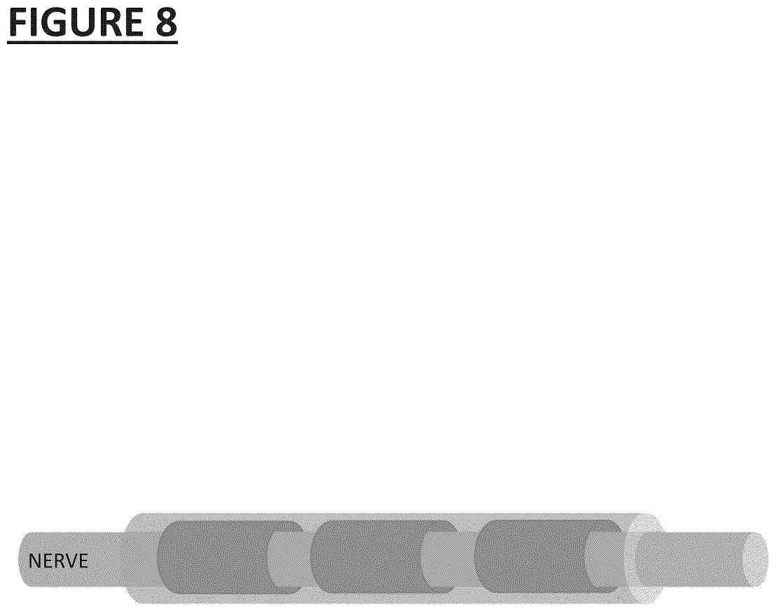

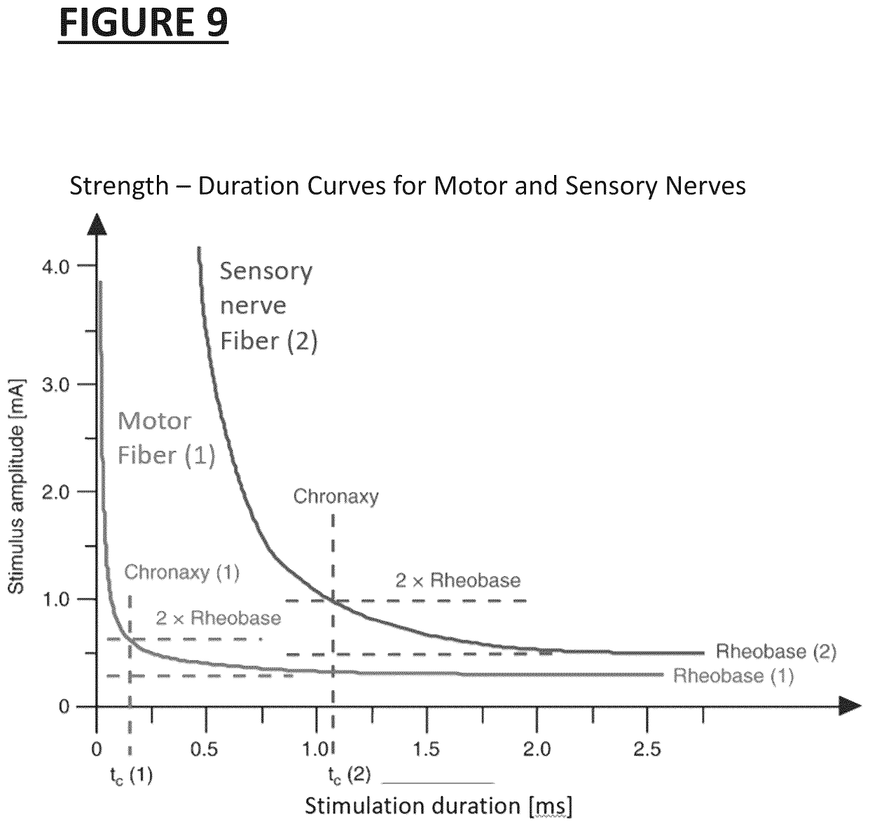

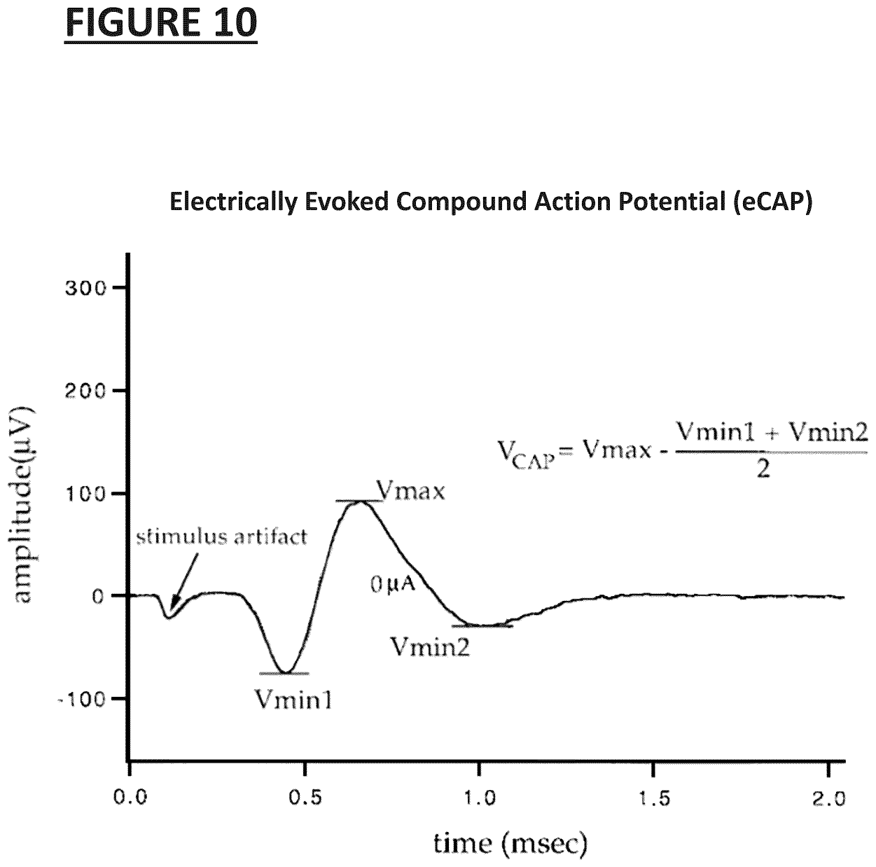

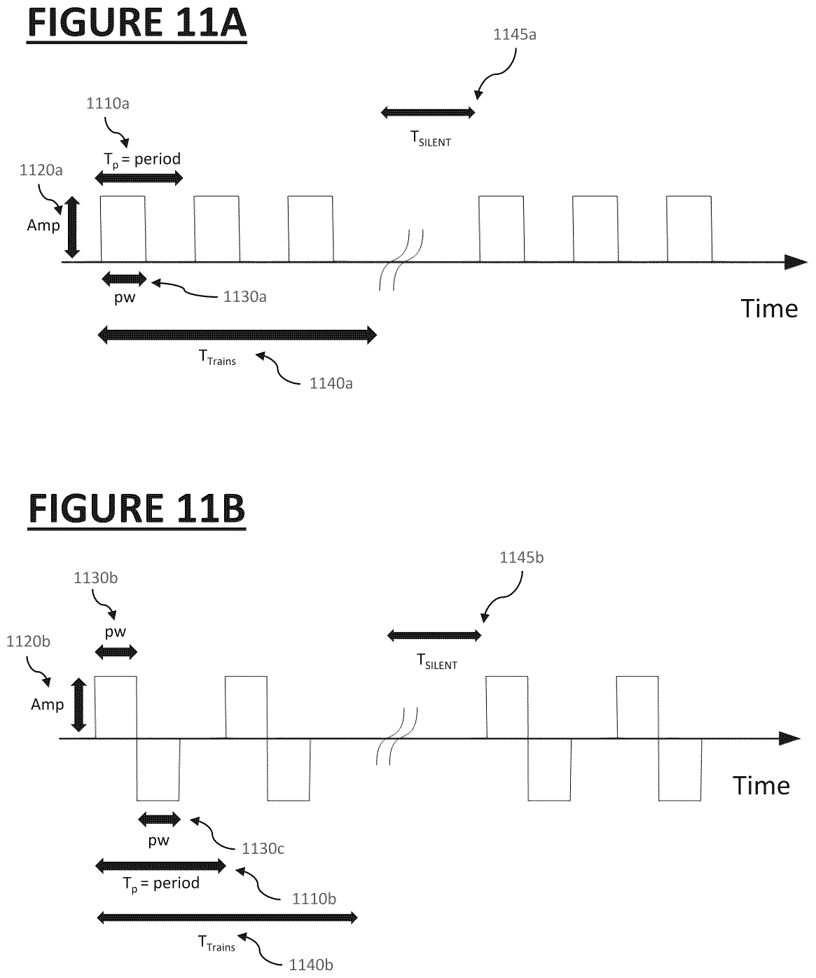

### DETAILED DESCRIPTION

“Sensory nerves” and “afferents” are terms used interchangeably and refer to nerves originating at peripheral organs, such as the diaphragm, and that carry information to the central nervous system.

“Motor nerves” and “efferents” are terms used interchangeably and refer to nerves originating at the central nervous system and carry information to the peripheral nervous system to, for example, produce excitation to the muscles, such as the diaphragm.

“Cuff electrode”, “electrode cuff”, and “electrode” are terms used interchangeably and refer to a device that can be placed into contact with target tissue, such as the phrenic nerve, while being coupled to electronics.

The term electrically evoked compound action potential (eCAP) represents the synchronous firing of a population of electrically stimulated nerve fibers. The eCAP can be directly recorded on a surgically exposed nerve trunk.

The cuff electrode for phrenic nerve stimulation can include four major components, which are:
a first body,
a second body,
an in-situ suturing mechanism, and
a performed lead.

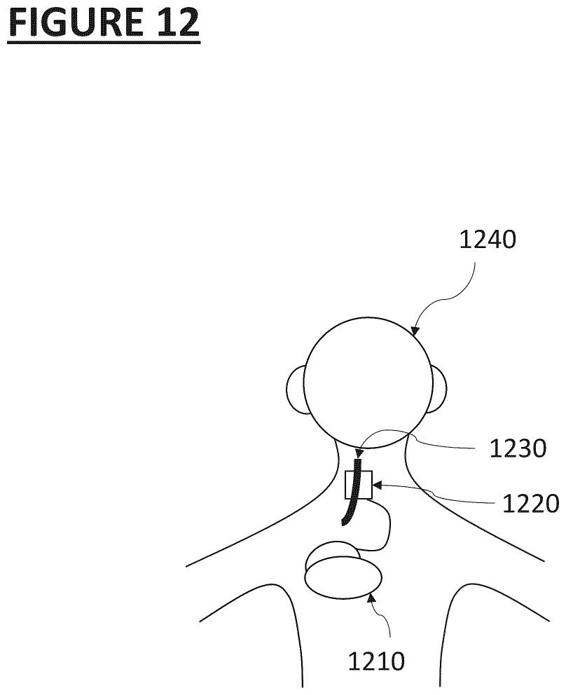

FIG. 12 shows the clinical configuration of the phrenic nerve monitoring and modulation system during its training phase. The system includes one or more sensors, an IPG 1210, an electrode cuff 1220 in connection with (e.g., surrounding) the phrenic nerve 1230 of a patient 1240.

The system of FIG. 12 may also include one or more sensors for the monitoring of the physiological signals from the patient. These sensors can include, but are not limited to, electrocardiogram (ECG) sensors, electromyogram (EMG) sensors, nerve monitors, inertial sensors such as accelerometers, auscultatory sensors and microphones, electrical impedance sensors, ultrasonic sensors, temperature sensors, pressure sensors, and microwave sensors.

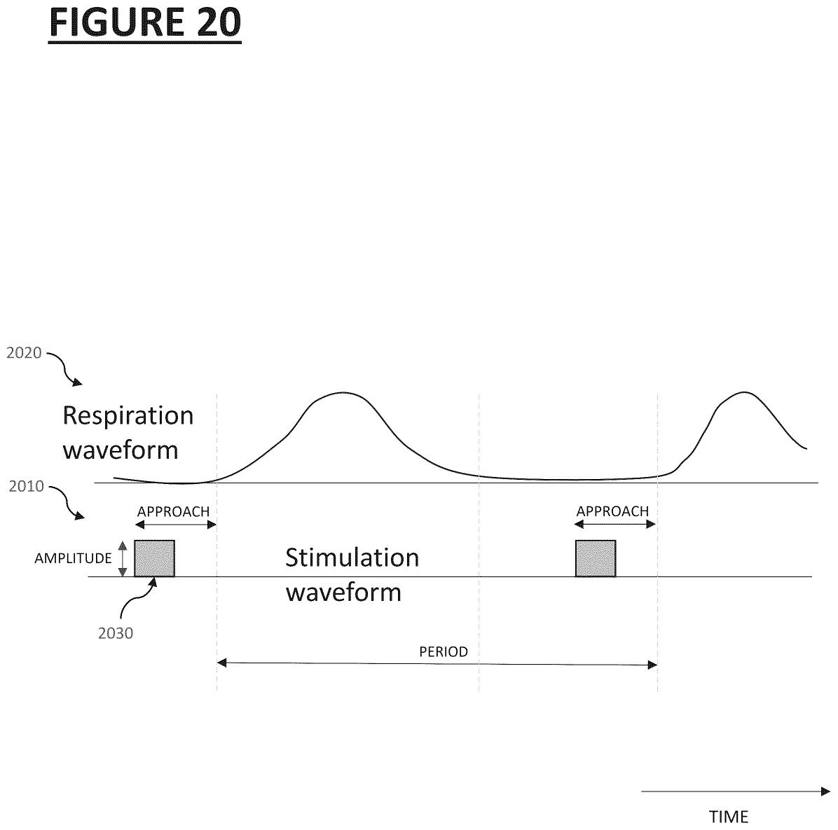

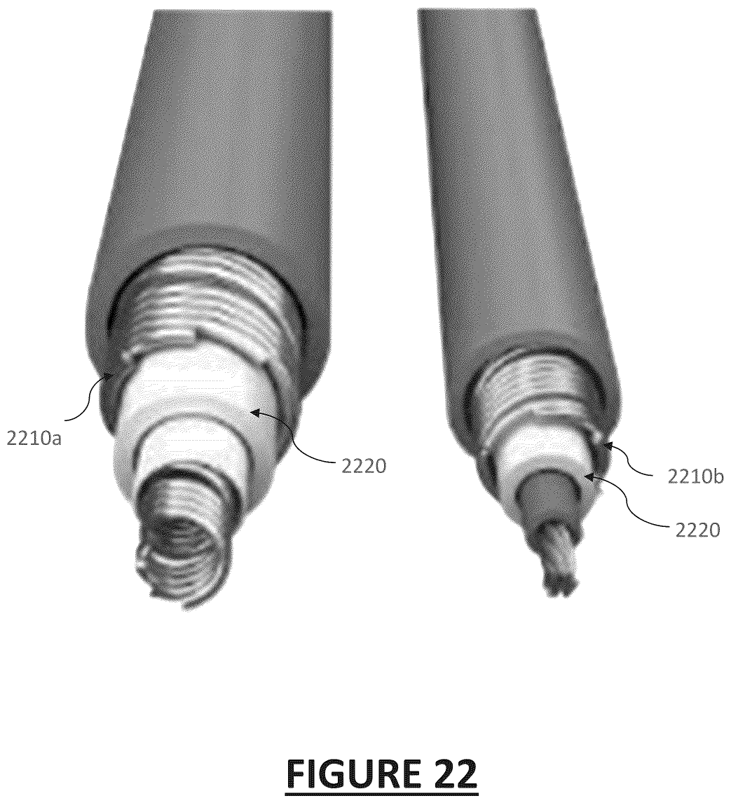

ECG sensors are used for the detection of the cardiac rhythms and eventual extraction of information, such as the heart rate. ECG signals can be obtained from the leads of the stimulator, as shown in FIG. 22, or from the sense electrodes placed on the stimulator, as shown in FIG. 20, which is referred to as leadless ECG. In all cases, the ECG signal is used to assess the cardiovascular component of the respiratory effect, as the beta sympathetic and para sympathetic efferent pathways from the central nervous system (CNS) directly affect the heart rate. ECG signals are usually in the frequency range of 0.05 Hz to 100 Hz, and can be detected using analog or digital circuits. ECG signals can also be used to determine heart rate that can indicate periodic breathing, sleep, and rest state.

Electromyogram (EMG) sensors detect the electrical signals resulting from muscle activity and muscle contractions. This information can be used for the detection of the contraction and relaxation of the diaphragm, rib muscles and muscles in the neck (axillary breathing muscles), and muscles of the upper airway. Accessory muscle use, defined as inspiratory contraction of the sternocleidomastoideoalene muscles, is associated with severe obstructive disease as well as hyperpnea and excessive effort associated with airway occlusion. The EMG frequency ranges vary from 0.01 Hz to 10 KHz, but the most useful and important frequency ranges are within the range from 50 to 150 Hz.

Examples of nerve monitors as discussed herein are for the detection of nerve activity in the nerves of interest, including, but not limited to, the vagus nerve, phrenic nerve, and the hypoglossal nerve. Amplitude of the signals measured are in the micro-Volt range and correspond to the action potentials. Since the nerve signals e.g. the evoked compound action potential (eCAP) generated by the motor neurons are easier to detect, they may be used in connection with one or more embodiments described herein, such as the detection of the activity of the phrenic nerve for the contraction of the diaphragm and application of the stimulation to excite the Nucleus Tractus Solitarius (NTS) which in turn activates Nucleus Anbiguous (NA) and increases the tone in Genioglossus Muscle via the activation of Cranial Nerve XII, also known as the Hypoglossal Nerve.

In certain example embodiments, the accelerometers can also be used as a type of sensor. Accelerometers are used for the detection of onset of sleep, sleep position, body motion during sleep and respiratory activity as well as respiratory effort. Patient activity and position during sleep is different during sleep state versus awake state. For example, during sleep, the torso of the patient can be positioned horizontally, which is detected by a 3D accelerometer monitoring a force which may be attributable to gravitational force. This information is used to detect the sleep state.

Some patients suffer from a condition known as positional sleep apnea, meaning that their apneic events occur more frequently during certain body positions during sleep. Accelerometers can sense the position of sleep, such as side, supine, or prone, and allow the therapy to be turned on or turned off depending on patient need. The ability to turn off the therapy when not needed can increase patient comfort and the tolerability of the therapy. Moreover, it can also prolong battery life of the implanted device.

In certain examples, accelerometers can be used to gather information about the respiratory activity or respiratory effort of the patients. An accelerometer that is within the implanted device can be used to pick up chest motion resulting from the respiratory activity. Use of an accelerometer can also allow the ability to distinguish between respiratory effort resulting in inhalation versus breathless activity, which would be an apnea. Accelerometers that are incorporated into the stimulation electrode and placed in the neck region can be used to gather the activity of upper airway muscles, and provide feedback for the therapy.

Microphones may be used to allow the detection of the sounds relating to the sleep. For example, if the patient is snoring, the implanted device interprets this as signs of successful inhalation and exhalation. Microphones can be placed on the chest or the neck region, and can be part of the implanted system or external. Analysis of the snoring sounds provides additional information that can be used by the treatment system. The frequency range of simple waveform snoring typically starts at 180 Hz and peaks at 300 Hz. The frequency range of complex waveform snoring typically begins at 60 to 130 Hz, with internal oscillations ranging up to 1 KHz. The higher the frequency, the greater the obstruction of the upper airways, which may be beneficial in certain example embodiments. Hence, in embodiment, the stimulator interprets the higher frequency content of the snoring sounds as it adjusts the stimulation parameters to reduce upper airway obstructions.

Pressure sensors can be used for the monitoring of the pressures in the chest or in the neck region, and can be used to determine if and when a contraction and relaxation begins. Pressure sensors placed in the neck region provide additional information regarding the muscle tone. Furthermore, the high frequency content of the pressure signals can be used as sound signals, to detect breathing or snoring sounds, as it was described above for the case of the microphones.

Ultrasonic sensors can be used in connection with certain example embodiments and may be used for the detection of distance between the sensor using the time-of-flight technique, where the sensors measure the time for a short burst produced by one sensor to arrive at the other sensor. This signal conveys two forms of information, namely the distance between the sensors and the media between the sensors. Distance between the sensors is proportional to the time that the transmitted ultrasound wave takes to travel from one transducer to the other. Attenuation that the received signal experiences is related to the nature of the tissue in between the transducers. For example, inhalation would expand the chest and increase distance between the transducers, which in turn would increase the time for the ultrasound pulse to travel from one transducer to the other. Transducers placed in the neck region can detect the muscle tone as changes in the signal attenuation. In other words, as the ultrasound signal would travel with less attenuation through muscles that are in contraction, which is a target of certain therapy techniques described herein.

Microwave signals that are generated by external devices can be used for the imaging of the tissues. The results can be conveyed to the stimulator, which in turn uses the information to further optimize an example treatment algorithm.

Additional sensors that are not listed above can be used for the detection of the tissue oxygen saturation, respiratory activity, nerve activity as well as muscle activity, and the like.

Sensors in the system can be located on the electrode cuff 1220 or directly attached to the patient 1240. Sensors may be embedded into the electrode cuff 1220 or attached to the electrode cuff 1220 depending on the type of sensor used. For example, pressure sensors may be embedded into the electrode cuff 1220 near the electrodes to ensure the phrenic nerve 1230 is making contact with the electrodes for stimulation.

Data from sensors and/or the electrode cuff 1220 can be read by a controller (e.g. which can include a microprocessor or other hardware processor). Given a type of input, the controller can produce (e.g., via a pattern generator that is provided as part of the controller) an output waveform for stimulation to the cuff electrode 1220. The pattern may include a train of pulses where the amplitude, pulse width, pulse frequency, and number of pulses are specified by the controller. Upon triggering by the controller, an output waveform for stimulation is generated. This is then delivered to the cuff electrode 1220 to be applied to the nerve.

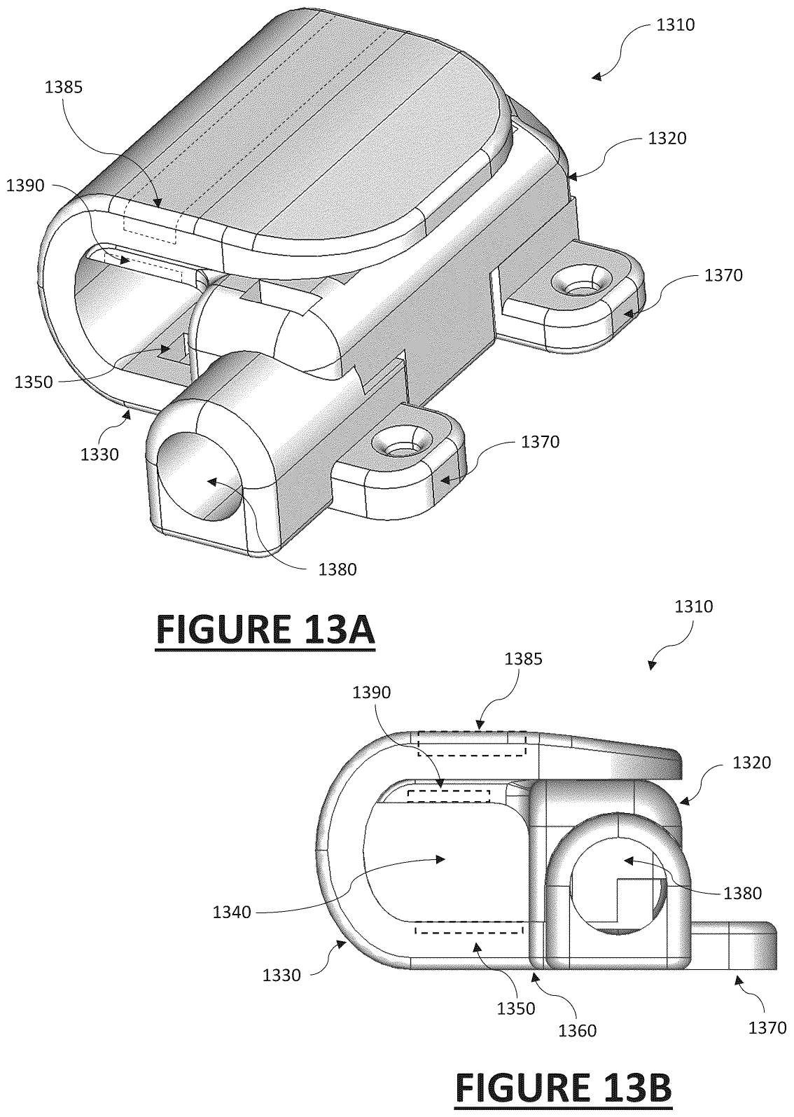

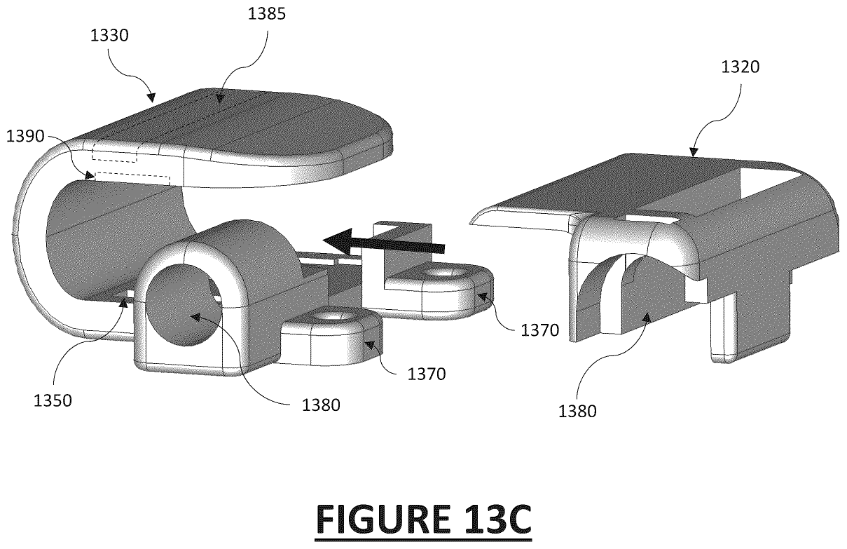

FIGS. 13A-13C illustrate different views of an electrode cuff to stimulate the phrenic nerve, according to an example of the present disclosure. As shown in FIG. 13A, the cuff electrode includes a cuff body 1310 including a first body 1320 and a second body 1330, which can be coupled together (as shown in FIG. 13A) to form an oblate lumen 1340 to accommodate the nerve and dissected fascia, especially in cases of nerve swelling. The first body 1320 and second body 1330 (shown separated in FIG. 13C) are both shaped in such a way that the side parallel to the major axis of the interior ellipsoidal space is flat, and the side perpendicular to the major axis of the interior ellipsoidal space is curved, forming an oblate ellipsoid when joined together. The second body's 1330 curved side is longer to overlap the first body 1320 and create an oblate ellipsoidal tunnel 1340 for the nerve to pass through, shown in FIG. 13B.

In some examples, the first body 1320 and second body 1330 may be bonded inflexibly on one side with a flexible opening on the opposite side. Material to construct the first body 1320 and second body 1330 of the cuff body may use one, or a combination, of silicone, polyethylene, polyester, polyether, polycarbonate, polypropylene, polystyrene, urethane, acrylic, epoxy, glass, rubber, thermoplastics, Delrin. Flexible material allows the phrenic nerve to not be overly constrained, limiting discomfort for the patient. In some examples, the cuff body may also have a rougher external material to reduce cuff migration. In some examples, the first body 1320 and/or the second body 1330 may be flexible single piece components.

As shown in FIG. 13A, electrodes 1350 may be embedded into the second body 1330 to orient electrodes 1350 on the bottom of the nerve. The phrenic nerve will naturally rest on the bottom of the cuff which minimizes the nerve capture threshold as the nerve will tend to sit against the electrodes. Additionally, electrodes on the second body 1330 prevent the first body 1320 from blocking the nerve from the electrodes if folded inside of the cuff 1310, due to the first body 1320 being shorter in length. The electrodes (e.g., those of the second body) can be formed out of metals (e.g., stainless steel, titanium, gold, platinum, or iridium), or an alloy of those (e.g., a metallic alloy), and/or a conductive polymer.

The cuff body 1310 may have flat external sides 1360 to fit into the surrounding anatomy of the phrenic nerve, lying against the belly of the anterior scalene muscle on the bottom (deep side) and against the posterior cervical fat pad on the top (superficial side).

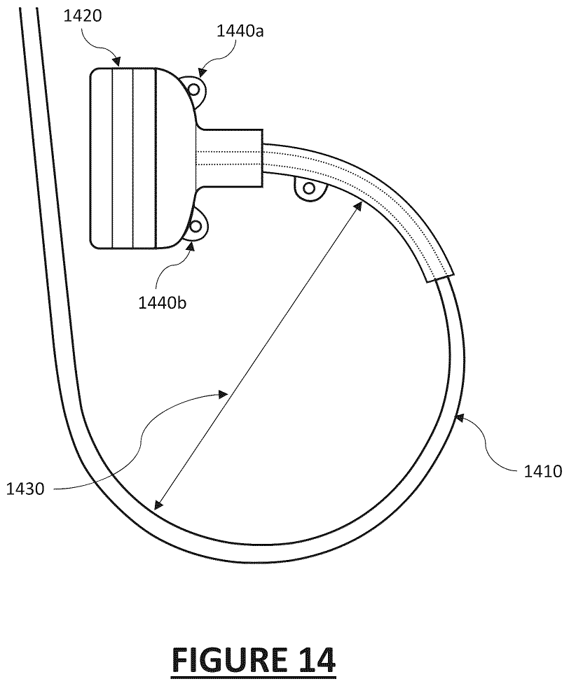

In some examples, cuff suture anchors 1370 can allow for anchoring the cuff near the phrenic nerve. Example embodiments shown in FIGS. 14-16 describe mechanisms for stabilization via sutures.

In some examples, the physical dimensions of the cuff electrode body can be, but are not limited to, 1 cm in the z-direction, in the range of 0.2 cm to 2 cm, and a 6 mm outside diameter, in the range of 2 to 10 mm. The inside measurements of the cuff electrode can be 1-3 mm for the minor axis and 2-4 mm for the major axis.

In addition to electrodes, the first body 1320 and second body 1330 can contain sensors 1385 1390 for sensing, signal conditioning, and stimulation of the nerve. Sensors 1385 1390 that may be embedded into the first body 1320 and second body 1330 include, but are not limited to, electrocardiogram (ECG) sensors, electromyogram (EMG) sensors, nerve monitors, inertial sensors such as accelerometers, auscultatory sensors and microphones, electrical impedance sensors, ultrasonic sensors, temperature sensors, pressure sensors, and microwave sensors. The benefits of including sensors 1385 1390 into the cuff body 1310 of the cuff electrode are similar to the reasons detailed above in FIG. 12. Additional cases may be to measure afferent traffic after and in response to stimulation. It may be also useful to sense the activity of local muscles, movement, or acceleration to ensure there is no interference with the nerve.

The lead connector or preformed lead insert 1380 can have different preformed shapes to optimize stabilization around the nerve's anatomy. Different embodiments of such preformed leads are described below.

FIG. 13C illustrates an exploded view of the described electrode cuff 1310 of FIG. 13A-13B, where the first body 1320 and second body 1330 are separated. In certain example embodiments, first body 1320 and second body 1330 can be connected via a hinge in order to connect the two pieces. In certain example embodiments, the hinge may be a polymer hinge.

In this embodiment, the phrenic nerve is mentioned as the target for stimulus. However, the vagus nerve and hypoglossal nerve may be stimulated as well in order to cure sleep disorders.

Turning to FIGS. 14-16, are different techniques and/or embodiments for routing the lead from an example cuff are described. In certain example embodiments, the lead may be pre-formed into a shape that fits into the anatomy of a patient. This approach may be in contrast to other approaches where the lead is unformed. Having an unformed lead can leave residual forces on the nerve and can overly rely on physician placement (which can increase the variability of placement of the cuff). In other examples, a generic shape like a sigmoid can be used. For the example leads shown in FIGS. 14-16, each of the shown leads may be connected to the example cuff shown in FIGS. 13A-13C.

FIG. 14 illustrates an embodiment of a preformed lead connected to a cuff electrode body (e.g., 1310 as shown in FIGS. 13A-13C) medially to minimize torque on the electrode cuff. In this example, a lead connector 1410 is connected to a cuff body 1420, where the lead 1410 is routed in a ¾ circle 1430 to fit into the surrounding anatomy of the phrenic nerve. Cuff suture anchors 1440a and 1440b (which may correspond to suture anchors 1370 from FIG. 13A according to certain example embodiments) can be located on the cuff body in order to stabilize the cuff body and ensure the phrenic nerve lies on the cuff electrodes. In some examples, suture anchors may be located on the lead wire to anchor the lead wire to the intermediate tendon of the omohyoid, the sternocleidomastoideole, or the ASM.

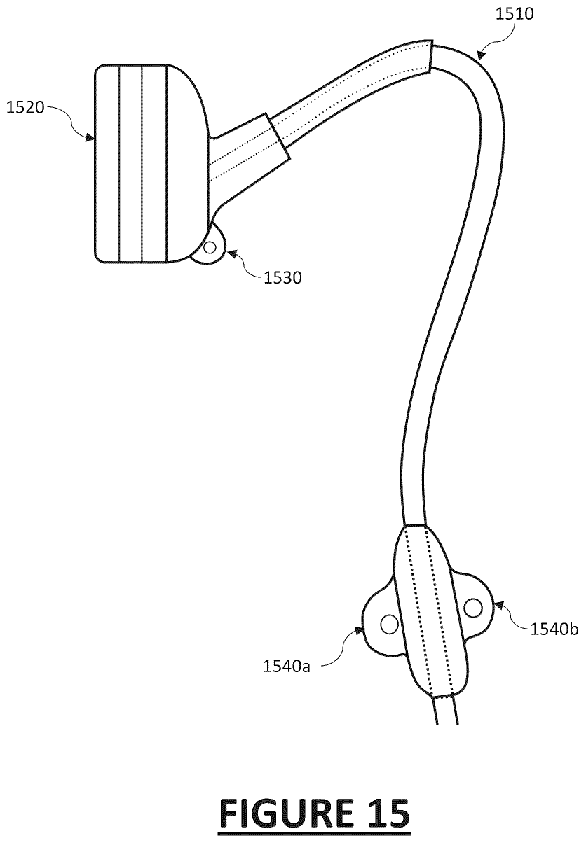

FIG. 15 illustrates a different embodiment of a preformed lead connected medially to a cuff electrode (e.g., 1310 as shown in FIGS. 13A-13C). The preformed lead 1510 exits the cuff body 1520 medially to minimize torque on the electrode cuff. In this embodiment, the preformed lead 1510 is routed medial and superior then inferior to avoid interaction with the laterally-located brachial plexus. Cuff suture anchors 1530 can be located on the cuff body in order to stabilize the cuff body and ensure the phrenic nerve lies on the cuff electrodes. In some examples, suture anchors 1540a and 1540b (which may correspond to suture anchors 1370 from FIG. 13A according to certain example embodiments) may be located on the lead wire to anchor the lead wire to the intermediate tendon of the omohyoid, the sternocleidomastoideole, or the medial anterior scalene muscle (ASM).

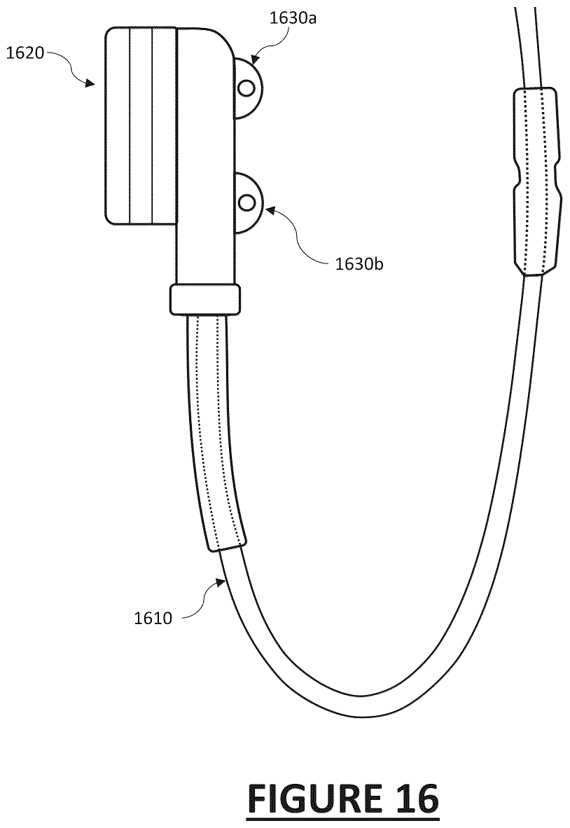

FIG. 16 illustrates a different embodiment of a preform lead 1610 routed through a cuff body 1620 in line with the cuff body 1620. FIG. 16 shows a preformed lead 1610 that exits the cuff body 1620 in-line, to allow the lead 1610 to run along the natural body tension lines and minimize cuff interaction with surrounding muscles. The curvature of the preformed lead 1610 allows the lead 1610 to be routed first superior, then lateral/medial (depending on the implant side), then inferior, minimizing off-tension line routing and preventing the lead 1610 from crossing itself. Cuff suture anchors 1630a and 1630b (which may correspond to suture anchors 1370 from FIG. 13A according to certain example embodiments) can be located on the cuff body in order to stabilize the cuff body 1620 and ensure the phrenic nerve lies on the cuff electrodes. In some examples, suture anchors may be attached to the ASM laterally or medially to the phrenic nerve, depending on the implant side. In some examples, lead anchoring may be attached to the sternocleidomastoideole and the intermediate tension of the omohyoid.

In some examples, the leads connected to the cuff body may be fully encapsulated in a flexible material, such as silicon. In other embodiments, the leads may be partially encapsulated or completely exposed for more flexibility of the leads. The lead may leave the electrode cuff parallel to the cuff, or at a 45-degree angle to fit to the surrounding anatomy. FIG. 16's preformed lead may leave the cuff electrode from the left or right side of the electrode cuff.

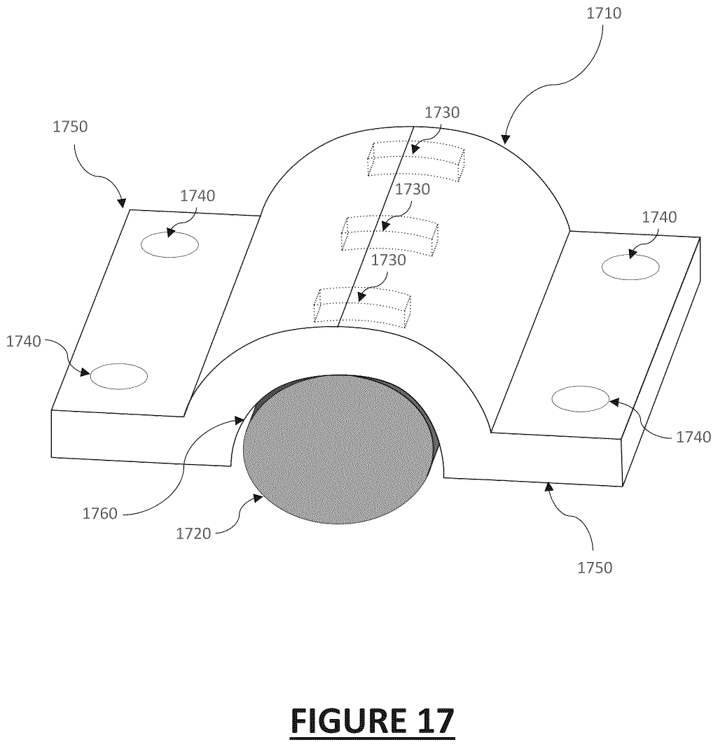

FIG. 17 is a different embodiment of a unilateral cuff electrode 1710 to stimulate the nerve 1720. FIG. 17 illustrates a one-sided cuff 1710 that lays over the nerve 1720. Electrodes 1730 are embedded into the bottom of the electrode cuff in the interior cuff space to contain the nerve 1720 and allow for the nerve 1720 to move side-to-side within the open space. The configuration of the electrodes 1730 is designed to prevent nerve dissection by being placed overtop the prevertebral fascia and may be sutured or otherwise adhered to the underlying muscle via one or more mechanisms per side, adjacent to the target nerve. Sutures 1740 can be built into the unilateral electrode cuff flaps 1750 to prevent the nerve 1720 from losing contact with the electrodes 1730. In some embodiments, sensors, such as pressure sensors, may be placed near the electrodes 1730 to ensure stimulation of the nerve 1720. Sutures may also extend from the flaps 1750 of the unilateral cuff electrode 1710, depending on the surrounding anatomy of the cuff electrode 1710.

Flaps 1750 extend from the semi-circular 1760 surrounding of the unilateral cuff electrode 1710 to adhere to the muscle near the nerve 1720. This allows the muscle to act as a wall entrapping the nerve once the flaps 1750 are sutured to the surrounding anatomy. In this embodiment, the semi-circular nerve channel 1760 may be 180° in arc to allow side-to-side movement of the nerve 1720. However, the semi-circular channel 1760 may extend further downwards to ensure the nerve does not slip out of the bottom of the channel, creating an arc with more than 180°.

In a different embodiment, the flaps 1750 may be removed from the unilateral cuff electrode body 1710 completely. In this embodiment, the flaps 1750 would be replaced with sutures extending from the electrode cuff body 1710 to mount the unilateral cuff electrode 1710 to the surrounding anatomy and adhere to the nearby muscle.

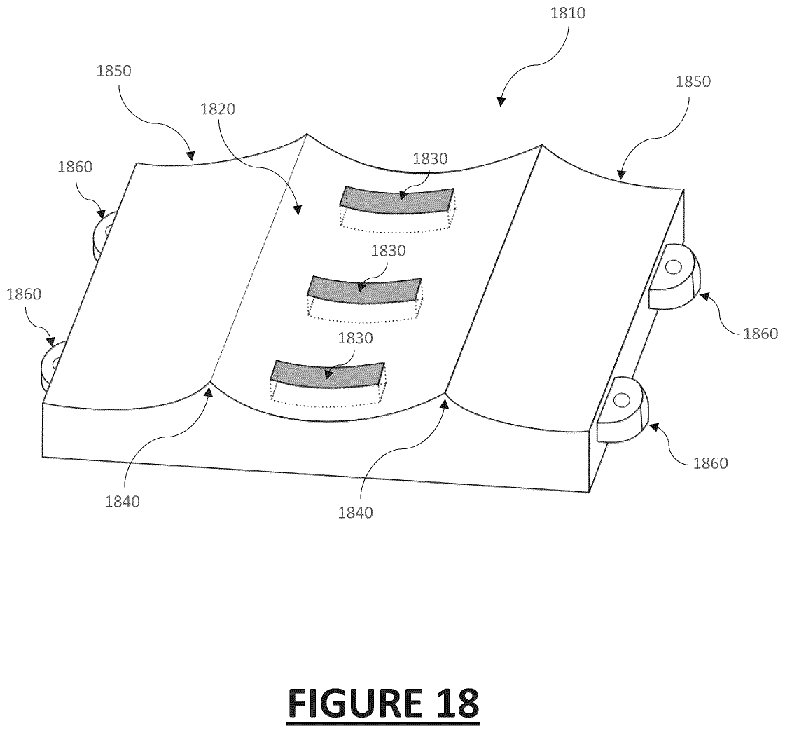

FIG. 18 is a different embodiment of a cuff electrode 1810, where the cuff body has an open-face design 1820. FIG. 18 is a cuff electrode 1810 with a one-sided configuration of electrode 1830 in which the nerve would lie between two raised portions 1840 of the electrode cuff 1810. To describe this embodiment differently, the electrode cuff 1810 is sectioned into three sections, where the outer two sections 1850 are slightly higher than the middle section 1820 resulting in a trough for the nerve to lie in. The electrodes 1830 are embedded into the lower trough 1820, so the electrodes 1830 can make secure contact with the nerve. The electrode cuff of this embodiment may be sutured 1860 or otherwise adhered to the underlying muscle via one or more mechanisms per side, adjacent to the phrenic nerve. In another example, sutures may be built into the outer flaps 1850 of the electrode cuff for stabilization of the nerve. To ensure the nerve does not move the arc of the trough 1820 may extend 45° or more degrees. Sensors may be placed where the nerve lies in order to detect if the nerve is not making contact with the electrodes 1830.

In some examples, there may be 1 or more contacts embedded into the cuff electrode body. In embodiments where multiple contacts are embedded, these contacts may be wired together or wired separately. When the contacts are wired together, the stimulation applied would be in the form of unipolar, meaning that all contacts will be at the same electrical potential with respect to the return electrode that is placed at a distance, which could be the metal casing of the implantable pulse generator (IPG) that is connected to the cuff electrode or another electrode. By wiring all contacts of the cuff electrode together, one eliminated the need for a multi conductor lead to be used between the IPG and the cuff electrode which in turn makes the lead softer and thinner.

If the contacts of the cuff electrode can be accessed separately, the one can drive them at different potentials, which in turn allows the delivery of multipolar stimulation, such as bipolar stimulation. Multipolar stimulation eliminates the need for a return electrode, and limits the spread of the electrical fields generated by the cuff electrode, which in turn prevents the excitation of non-target organs, as it would be in the case of unipolar stimulation. Another advantage of the contacts that are wired separately is the ability to address them individually, which may be necessary if strategies such as nerve blocking is to be used to achieve unidirectional stimulation of the target nerve, such as the phrenic nerve.

The electrodes embedded into the cuff electrode may be the entire length of the electrode cuff or shorter. One may want to change the length of the electrode depending on the amount of stimulation needed to be applied to the nerve. The targeted nerve bundle may also change the length of the electrode.

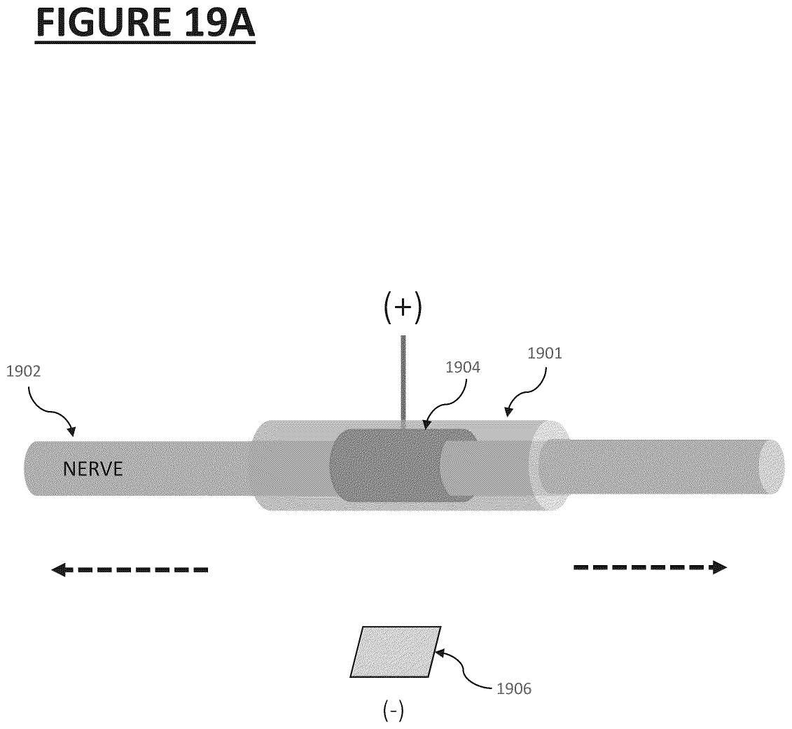

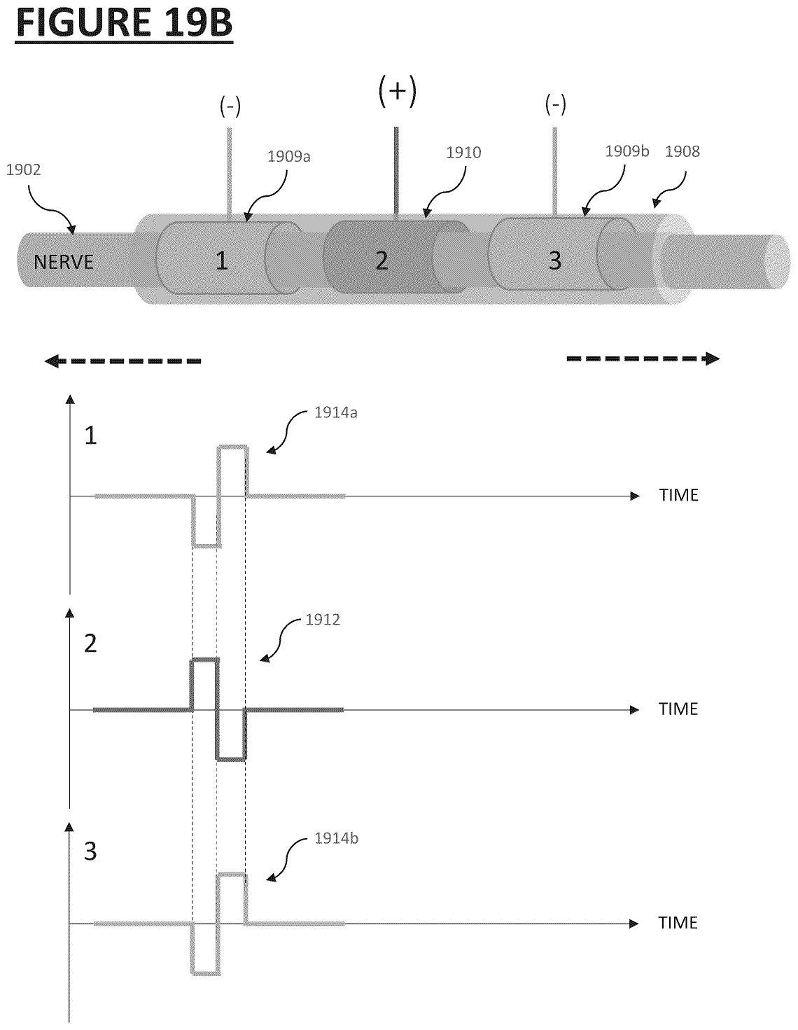

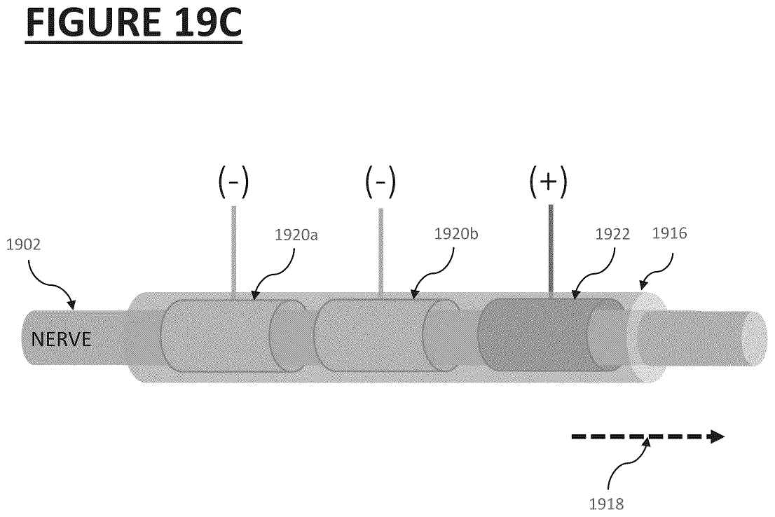

FIG. 19A illustrates a monopolar cuff electrode 1901 for bidirectional stimulation. Monopolar cuff electrode 1901, as shown in FIG. 19A, may be designed to provide stimulation to the target tissue, usually the nerve 1902, by a single contact 1904 where the return electrode 1906 is located at a distance. The return electrode 1906 for an implanted system is usually the case of the stimulator, although it can be a different electrode as well. Monopolar electrodes 1901 have the advantage of being simple to design and implant while also being smaller in size. However, they suffer from the disadvantage of not being specific for stimulation or sensing, meaning that inadvertent stimulation of the tissue between the cuff electrode and the return electrode, e.g., the implanted device is possible. Similarly, it is possible to sense signals that are not generated by the target nerve 1902 but are caused by the sources that are in the vicinity of the monopolar cuff electrode 1901 or near the return electrode 1906. Furthermore, any stimulation that is applied to the monopolar cuff electrode 1901 will generate excitation in the target nerve 1902 which will travel bidirectionally, preventing the ability to select the excitation of the afferent or efferent nerve fibers only. However, due to their simplicity, the monopolar cuff electrodes 1901 can be used in accordance with certain examples embodiments discussed herein.

An example tripolar cuff electrode 1908 is shown in FIG. 19B. Such electrodes are designed to provide stimulation to the target tissue, usually a nerve 1902, through multiple contacts. In the case that the tripolar cuff electrode 1908 contains three electrodes 1909a, 1909b, 1910, stimulation is generated such that the two outer electrodes (which may also be termed a contact of an electrode herein) 1909a 1909c carry an electrical potential that is opposite of the potential of the central electrode 1910. For example, when the central electrode 1910 is held at a positive potential, the outer electrodes 1909a and 1909b are held at a potential that is equal in amplitude, but opposite in sign, i.e., negative. This type of design concentrates the stimulation to the nerve 1902 that is being targeted and reduces the possibility of the stimulation of unintended tissues. Stimulation that is delivered to the contacts can be monophasic or biphasic. In monophasic stimulation, the waveform that is applied to each contact has a single phase, and it is not alternated. In a biphasic stimulation, the waveform has two phases and alternates once for each cycle. The use of biphasic stimulation can be more advantageous since it reduces the chances of electrode corrosion and the potential damage to the target tissue by limiting the free radicals and excess of ions that are being gathered around the contacts.

Tripolar cuff electrodes can be used for bi-directional or unidirectional stimulation. When bidirectional stimulation of a nerve is desired, electrical potentials as indicated in FIG. 19B are applied to the contacts of the tripolar cuff electrodes, where the inner contact 1910 is held at a potential that is opposite (1912) of the potential of the potential of the outer contacts 1909a and 1909b (1914a). Furthermore, the bidirectional stimulation of the nerve is generally initiated by the negative phase at the outer contacts 1909a 1909b and the positive phase at the inner contact 1910, which is further illustrated in FIG. 19B. The resulting action potential would travel in either direction, capturing both the afferent and efferent nerves.

FIG. 19C illustrates a unidirectional cuff electrode 1916 that provides unidirectional firing for stimulating a nerve 1902. For the generation of unidirectional stimulation, two contacts 1920a 1920b on the non-traveling direction of a three-contact electrode are kept at a negative potential while delivering a positive pulse to the contact 1922 on the traveling direction 1918. This pattern allows the depolarization of the nerve 1902 on the travel direction 1918 while keeping the segments of the nerve 1902 on the non-travel direction hyperpolarized. Unidirectional stimulation of the nerve 1902 allows the selective capture of afferent or efferent nerves in a bundle that the cuff electrode 1916 surrounds while providing nerve blocking.

Stimulation from electrodes can be applied in different waveform patterns. FIG. 20 illustrates a stimulation waveform 2010 based on a respiration waveform 2020. The respiration waveform shows the volume of air in the lungs as a patient inhales and exhales. In the shown pattern, stimulation is applied during the pre-inhalation phase, where the block 2030 represents stimulation time. In other examples, stimulation may be applied continuously, continuously except during inhalation, and only during the inhalation time, but is not limited to these stimulation patterns.

The lead connecting the cuff electrode to the IPG may be built using different design methodologies.

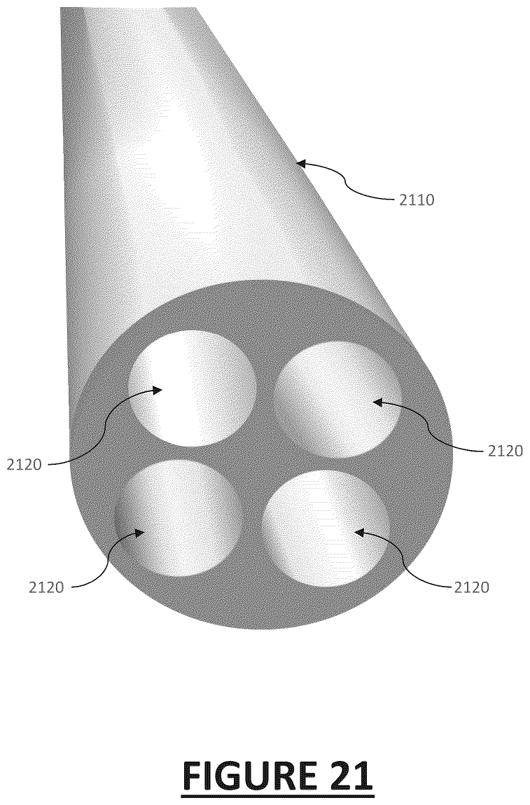

FIG. 21 shows a multi-lumen design where the wires connected to different contacts in the cuff electrode travel through the lead 2110 in different lumens 2120, i.e., multi-lumen design.

FIG. 22 shows a concentric conductor lead design where the conductors 2210a 2210b are separated by a layer of insulator 2220 in between them.

FIG. 23 shows a multi conductor spiral design where each conductor has the same pitch and radius, but are still insulated 2330 from one another. The multi conductor design alternates in conductors, that is, a conductor 2310 connected to contact #1 will be coiled between a conductor 2320 that is connected to contact #2, and so on, with inside insulation 2330 between the conductors. In this embodiment, there may also be outside insulator 2340 to prevent damage to the conductors 2310 2320.

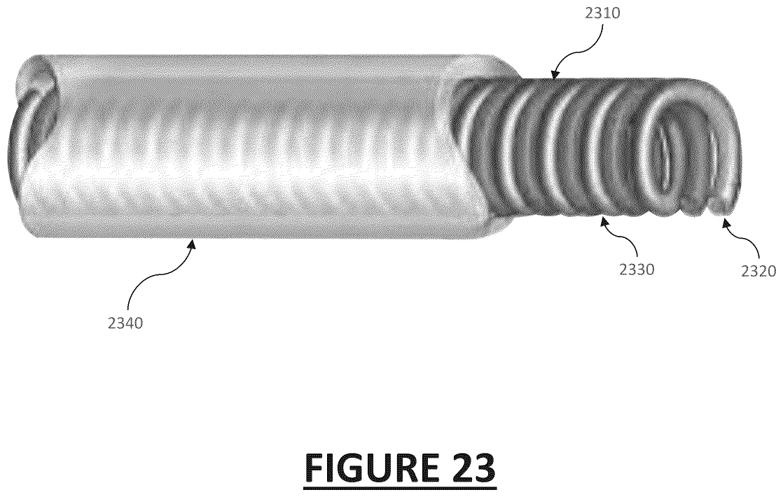

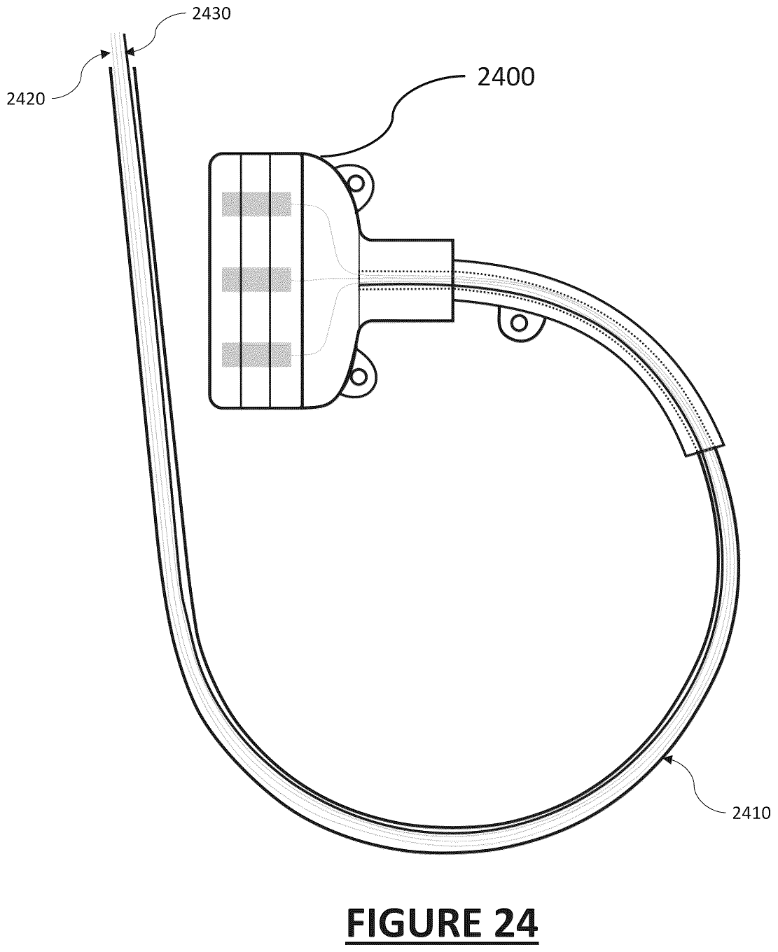

A lead 2410 that is connected to the cuff electrode 2400 may have a preformed shape to reduce the stress on the nerve resulting from the implant location and patient motion. The preformed shape can be induced on the lead by different methods. In one embodiment, the encapsulation of the lead is thermoformed, hence it retains its shape due to the structure of the enclosure. In another embodiment, the shape of the lead 2410 is due to a tension string 2430, as illustrated in FIG. 24. In this case, the lead 2410 has a lumen containing the conductors 2420 as well as a tension string 2430. By adjusting the tension of the tension string 2430, the curvature of the lead 2410 can be changed.

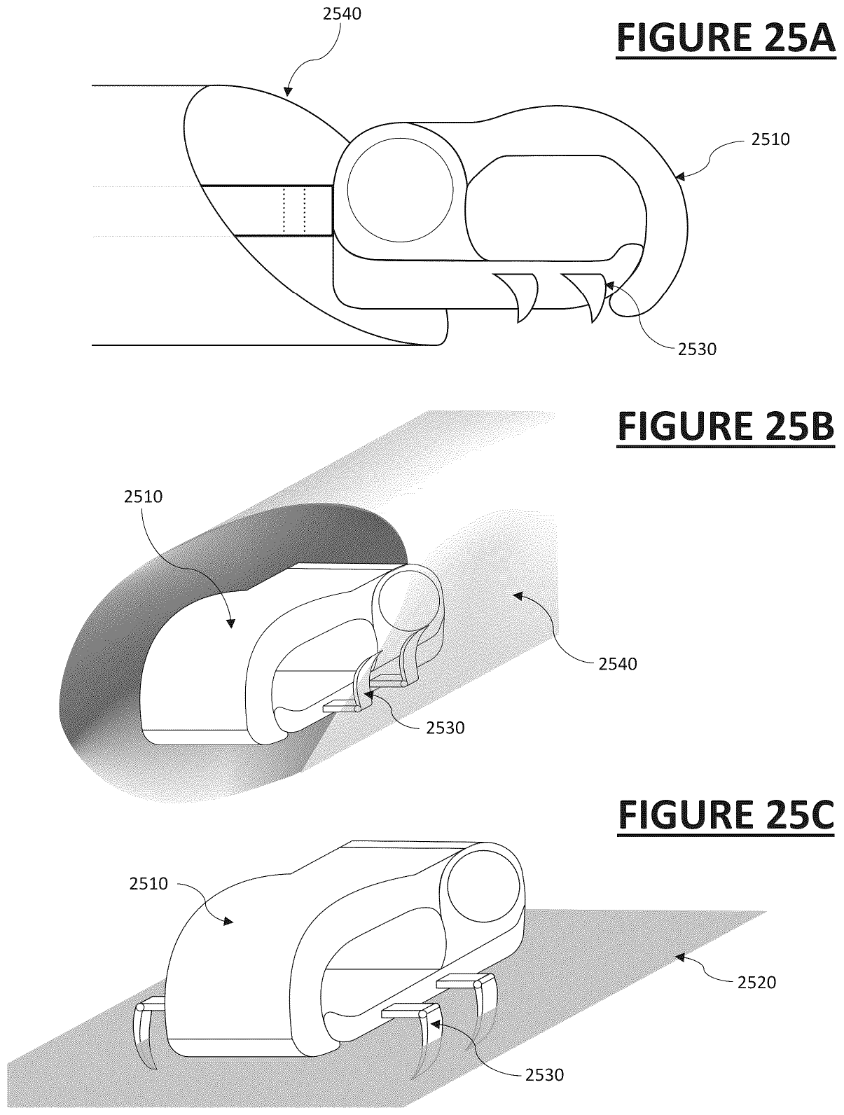

Fixation of the electrode to the muscle can be accomplished by different methods. For example, FIG. 13A shows an embodiment where the fixation may be provided via suture holes 1370. FIGS. 25A-25C show another embodiment where the fixation of an electrode cuff 2510 to muscle 2520 is achieved by spring-loaded hooks 2530. FIG. 25B shows the electrode cuff 2510 before deployment, where the hooks 2530 are kept upright using the sleeve 2540. Once the deployment is complete, then the sleeve 2540 is pulled back and the spring-loaded looks 2530 are released to penetrate into muscle 2520 as shown in FIG. 25C. The distal end of the sleeve 2540 has a slanted cut to allow the opening of the cuff electrode 2540 by lifting the top part for the placement of the phrenic nerve.

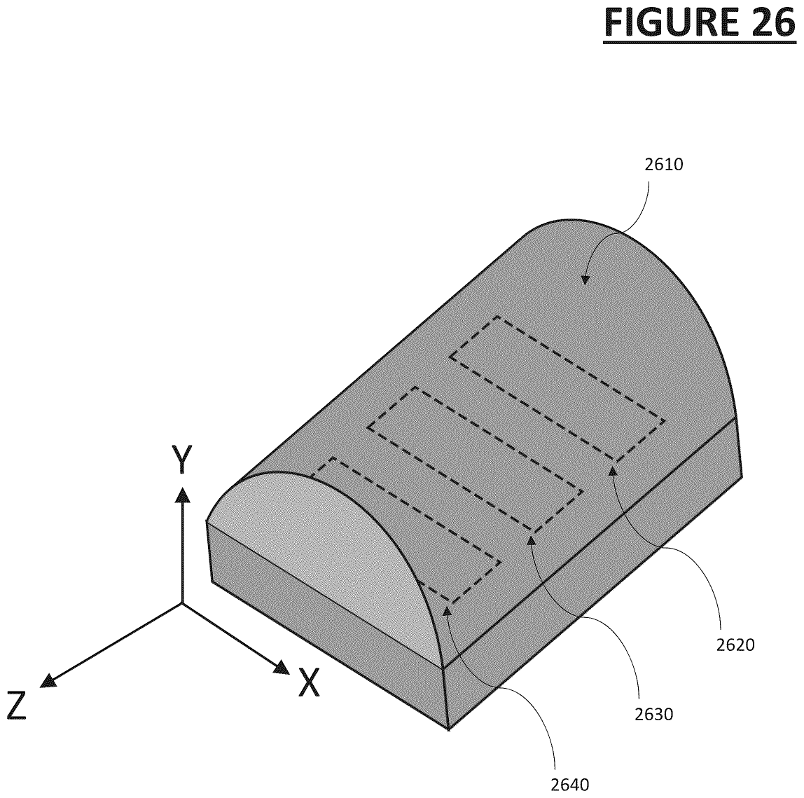

Contacts that are within the cuff electrode can be located so as to improve (e.g., maximize) the chances of capturing the nerve that is going through the cuff electrode. An example of placement of the contacts 2620, 2630, and 2640 in the cuff electrode 2610 is shown in FIG. 26. Contacts are formed using metals, titanium or alloys such as titanium-iridium. Furthermore, the contacts are placed on the flat base of the cuff electrode, and they are flush on the bottom surface, meaning that they do not protrude into the inner space of the cuff electrode. And finally, the size of the contacts as well as the spacing between them were determined to optimize the resulting electrical field. In the present application, the optimal electrical field is the one that maximizes the chances of stimulating the nerve that is residing within the cuff electrode while confining the electrical field within the cuff electrode to prevent the stimulation of non-targeted tissues.

In order to stimulate the nerve, electrical potentials are applied to the contacts 2620, 2630, and 2640. Although many stimulation configurations and patterns are possible, an example is provided where positive potentials are applied to the outer contracts, e.g., 2620 and 2640, while the middle contact 2630 is maintained at a negative potential.

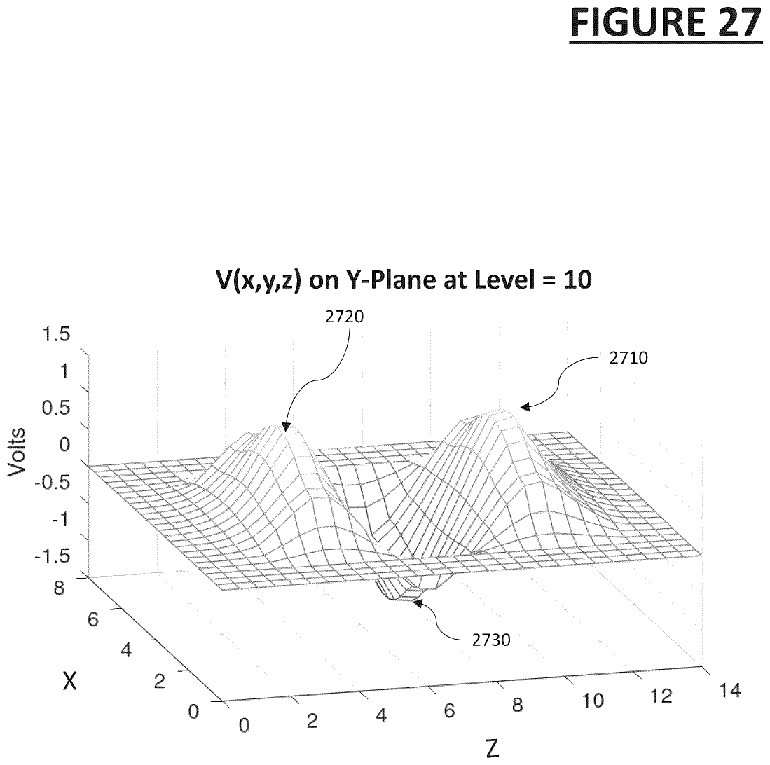

FIG. 27 shows voltages present inside the cuff electrode that is parallel to the flat base of the electrode. As it can be seen from the voltage map, two positive voltage areas were observed—2710 and 2720. These correspond to the positions above the contacts that are receiving positive excitation, e.g., 2620 and 2640 in FIG. 26. Similarly, the negative peak 2730 in FIG. 27 occurs above the middle contact 2630 of FIG. 26.

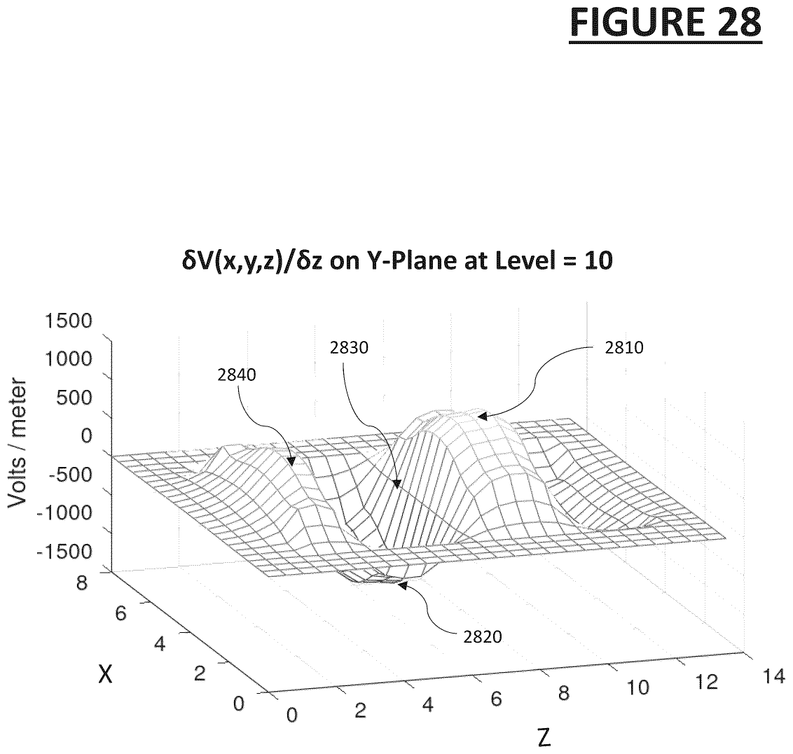

FIG. 28 shows the gradient of voltages in Z-direction, i.e., ∂V(x,y,z)/∂z, or the electrical field in Z-direction, E_{z}(x,y,z). As it can be seen from FIG. 28, the maximum of E_{z} (2810) occurs between the contacts 2620 and 2630 of FIG. 26, while the minimum E_{z} (2820) of FIG. 28 occurs between the between the contacts 2630 and 2640 of FIG. 26. Another minor positive peak 2840 is also generated. Furthermore, it should be noticed that the maximum positive slope on E_{z} (2830) occurs above the center contact 2630 of FIG. 26.

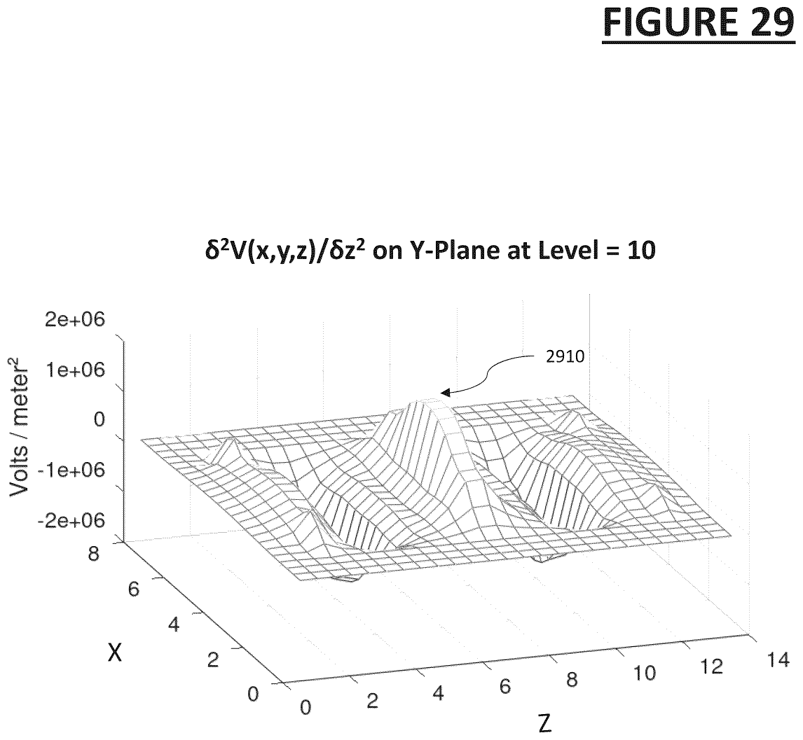

FIG. 29 shows the gradient of E(x,y,z) in Z-direction, or ∂²V(x,y,z)/∂z². It will be appreciated that the higher the gradient of the applied E-field along the axis of the axon, the more likely the nerve will depolarize. Since the nerve is aligned with the Z-axis of the cuff electrode, as shown in FIG. 26, it can be desirable to maximize the gradient of E-field along the Z-axis, i.e. ∂E(x,y,z)/∂z or ∂²V(x,y,z)/∂z². As seen from FIG. 29, the maximum of the gradient of E-field along the Z-axis (2910), corresponding the maximum slope 2830 of FIG. 28, occurs above the central contact 2630 of FIG. 26. By arranging the contacts 2620, 2630, and 2640 of the cuff electrode 2610 as shown in FIG. 26, and applying the potentials in order of positive (2620), negative (2630), positive (2640), the electric field gradient that is shown in FIG. 26 was obtained. This arrangement has two main advantages:

First, it produces the maximum positive peak E-field gradient along the axis of the nerve in the center of the cuff electrode, which assures that the most likely target to be captured is the nerve that is residing within the cuff electrode. The second advantage of the arrangement is that the E-field gradient is mostly negative near the edges and then quickly vanishes outside of the cuff electrode, which in turn prevents the inadvertent stimulation of non-target tissues outside of the cuff electrode. Hence the design forms a safe and effective device.

A close observation of FIG. 28 would indicate that the electrical field intensity, which is also the gradient of the voltage (∂V(x,y,z)/∂z) is not symmetrical in the Z-direction. This is due to a slight offsetting of the positions of the contacts in the cuff electrode, which is described below.

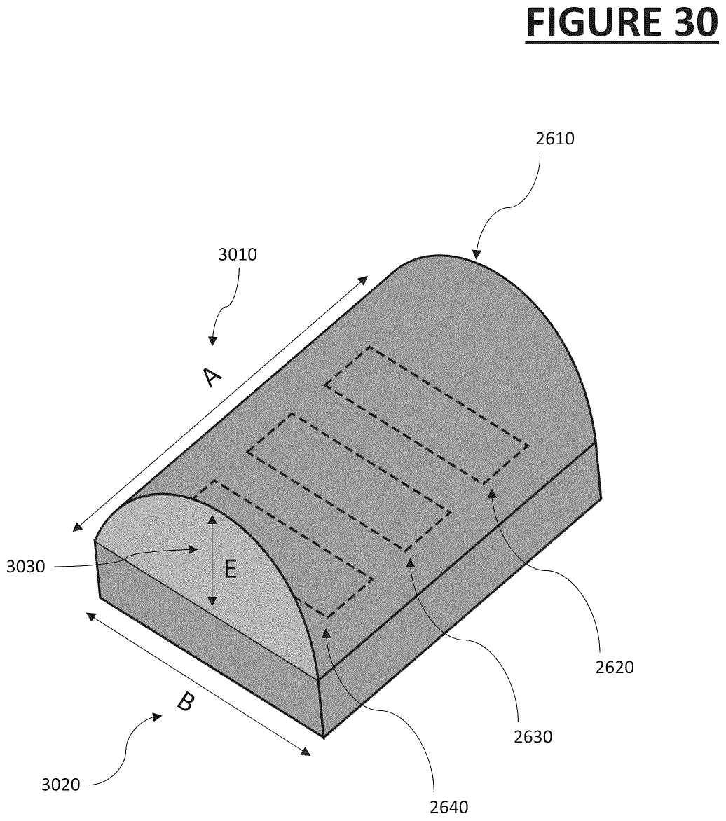

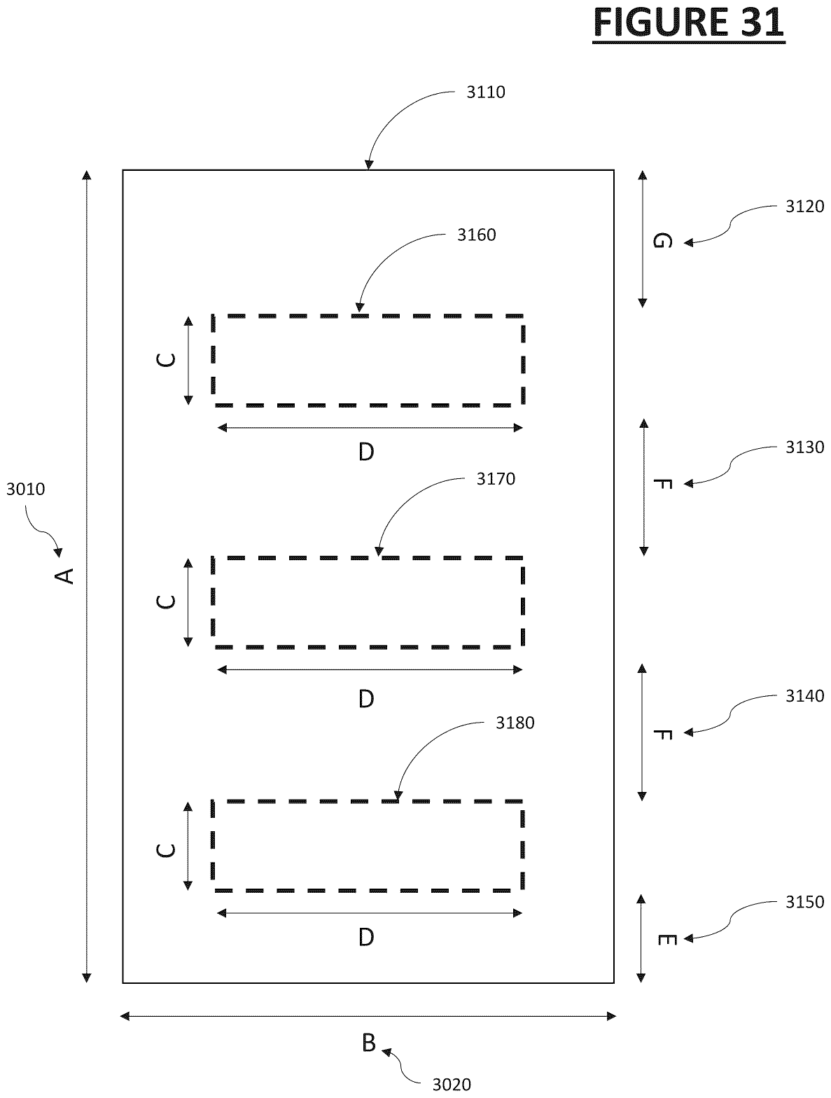

FIGS. 30-31 show example dimensions of a cuff electrode-including inside of the cavity. Example dimensions are listed below:

The interior height 3030 of the cuff is 0.5-0.75× the interior width of the cuff. The contact space from the edge of the cuff is 0.5-1.0× the interelectrode spacing. The anchor spacing is 0.5-1.0× the length of the inner cuff, The anchors are on one side of the cuff. The inside of the cuff is square except for a rounded outer flap (this is to address the current design which has a flat inner side and flat top and bottom). Electrode length is 0.5-0.7× the cuff inner width (previously called the major axis when the cross section was described as elliptical), and/or contact spacing is in a range of 0.6 to 0.9 times the height of the inner cuff for full nerve penetration of the E-field.

An electrode base is shown in FIG. 31. The base 3110 includes length A 3010 in a first direction (e.g., Z direction) and a length B 3020 in a second direction (e.g., X direction). Electrodes 3160, 3170, and 3180 can all have the same dimensions of C×D and they are separated by an F distance away from each other (3130 and 3140). However, electrode 3180 is only E distance 3150 away from the edge of the base while electrode 3160 is G distance 3120 from the edge of the cuff base. This offset is responsible for the non-symmetry of the electrical field shown in FIG. 28 where the major positive peak 2810 is stronger than the minor positive peak 2840, which in turn results in the steepest slope 2830 to form in the center of the cuff. FIG. 31 further illustrates that the maximum positive peak of E-field gradient (∂²V(x,y,z)/∂z²) 2910 occurs in the center of the cuff electrode. Since the positive E-field gradient (∂²V (x,y,z)/∂z²) is required for capturing a nerve, it is advantageous to have the positive peak at the center of the cuff electrode, which is in turn accomplished by the special positioning of the contacts of the cuff electrode.

While at least one exemplary embodiment of the present invention(s) is disclosed herein, it should be understood that modifications, substitutions and alternatives may be apparent to one of ordinary skill in the art and can be made without departing from the scope of this disclosure. This disclosure is intended to cover any adaptations or variations of the exemplary embodiment(s). In addition, in this disclosure, the terms “comprise” or “comprising” do not exclude other elements or steps, the terms “a” or “one” do not exclude a plural number, and the term “or” means either or both, unless the disclosure states otherwise. Furthermore, characteristics or steps which have been described may also be used in combination with other characteristics or steps and in any order unless the disclosure or context suggests otherwise. This disclosure hereby incorporates by reference the complete disclosure of any patent or application from which it claims benefit or priority.

### REFERENCE NUMBERS

0810 Nerve
0820 Electrode
0830 Electrode
0840 Electrode
0850 Cuff Electrode
1110a Tp=Period (11A)
1110b Tp=Period (11B)
1120a Amplitude (11A)
1120b Amplitude (11A)
1130a Pulse Width (11A)
1130b Pulse Width (11B Positive)
1130c Pulse Width (11A Negative)
1140a T Train (11A)
1140b T Train (11B)
1145a T Silent (11A)
1145b T Silent (11B)
1210 IPG
1220 Electrode Cuff
1230 Phrenic Nerve
1240 Patient
1310 Cuff Body
1320 First body
1330 Second body
1340 Oblate lumen
1350 Electrodes
1350
1360 Flat External Sides
1370a Cuff Suture Anchors
1370b Suture
1380 Preformed Lead Insert
1385 Sensor
1390 Sensor
1410 Lead Connector
1420 Cuff Body
1430 Lead Body Shape (¾ circle)
1440a Cuff Suture Anchor
1440b Cuff Suture Anchor
1510 Preformed Lead
1520 Cuff Body
1530 Cuff Suture Anchors
1540a Lead Suture Anchor
1540b Lead Suture Anchor
1610 Preformed Lead
1620 Cuff Body
1630a Cuff Suture Anchor
1630b Cuff Suture Anchor
1710 Unilateral Cuff Electrode
1720 Nerve
1730 Electrodes
1740 Suture Holes
1750 Unilateral Electrode Cuff Flaps
1760 Preformed Lead Shape (semi-circular)
1810 Cuff Electrode
1820 Open-Face Cuff Surface
1830 Electrode Configuration
1840 Raised Portions of the Cuff Body
1850 Outer Two Sections of the Cuff Body
1860 Sutures
1901 Monopolar Cuff Electrode
1902 Nerve
1904 Single Contact
1906 Return Electrode
1908
1908 Tripolar Cuff Electrodes
1909a Outer Electrode (−)
1909b Outer Electrode (−)
1910 Inner Electrode (+)
1912 Inner Electrode (+) Stimulation Graph
1914a Outer Electrode (−) Stimulation Graph
1914b Outer Electrode (−) Stimulation Graph
1916 Cuff Electrode
1918 Stimulation Travel Direction
1920a Outer Electrode (−)
1920b Inner Electrode (−)
1922 Outer Electrode (+)
2010 Stimulation Waveform
2020 Respiration Waveform
2030 Stimulation Time
2110 Multi-Lumen Lead
2120 Lumens
2210a Conductor
2210b Conductor
2220 Layer of Insulation
2310 Conductor
2320 Conductor
2330 Insulation
2340 Outside Insulation
2410 Preformed Lead
2420 Conductors
2430 Tension String
2510 Electrode Cuff
2520 Muscle
2530 Spring-Loaded Hooks
2540 Sleeve
2610 Cuff Electrode
2620 Contact
2620 Contact
2640 Contact
2710 Positive Voltage Peak
2720 Positive Voltage Peak
2730 Negative Voltage Peak
2810 Maximum of EZ (Most Positive Voltage Peak)
2820 Minimum of EZ
2830 Maximum Positive Slope on EZ
2840 Positive Voltage Peak
2910 Maximum of the Gradient of E-Field Along the Z-Axis
3010 Base Width (B)
3020 Base Length (A)
3030 Interior Height of the Cuff
3110 Base
3120 G Distance
3130 F Distance
3140 F Distance
3150 E Distance (asymmetry)
3160 Electrode
3170 Electrode
3180 Electrode

### Additional Embodiments

The following are additional example embodiments.

A1: An electrode cuff comprising: a second body including a bottom panel, an upper panel, and a connecting bridge joined to a first edge of the bottom panel and a first edge of the upper panel, wherein the upper panel is a cantilever extending from the connecting bridge and to a second edge of the upper panel opposite to the first edge; wherein the inner surfaces of the bottom panel, the upper panel, and the connecting bridge define a passage, wherein the passage is configured to receive a nerve through a gap between the second edge of the upper panel and the bottom panel; a first body configured to engage a side of the bottom panel wherein the first body forms a side of the passage when the first body is engaged with the side of the bottom panel; and electrodes in at least one of the inner surfaces of the bottom panel, the upper panel, or the connecting bridge, wherein the electrodes are configured to electrically stimulate a nerve with the passage. OR, A phrenic nerve stimulation and sensing device (also called electrode cuff in some examples) comprising: a first body with inner and outer surfaces in a form of an ellipsoidal dome; a second body with a flat inner surface and a flat outer surface where the inner surface contains one or more electrodes; electrodes that are located in the second body and oriented towards a passage formed by an assembly of the first body and the second body; an in-situ suturing mechanism for fixation to muscle; and a preformed lead conductively connected to electrodes in the second body.

A2: The electrode cuff of A1, wherein the second body and the first body are each rigid single piece components.

A3: The electrode cuff of A1 or A2, further comprising a hollow sleeve attached to the bottom panel configured to receive a conductive lead conductively attached to the electrodes.

A4: The electrode cuff of A3, wherein a main axis of the main passage is parallel to a main axis of the hollow sleeve.

A5: The electrode cuff of any of A1 to A4, wherein the passage is in cross section a curvilinear ellipse.

A6: The electrode cuff of any of A1 to A5, further comprising a hinge connecting the first body and the second body.

A7: The electrode cuff of any of A1 to A6, wherein the second body includes suture holes for passive fixation to underlying muscle tissue.

A8: The electrode cuff of any of A1 to A7, further comprising biased hooks on the second body, wherein the biased hoods are configured for active fixation to underlying muscle tissue.

A9: The electrode cuff of any of A1 to A8, wherein the electrodes on the second body are formed of stainless steel, titanium, gold, platinum, or iridium, or an alloy of those.

A10: The electrode cuff of any of A1 to A9, wherein the first body and the second body are formed of a non-conductive material, such as silicone, polyester, polyether, polycarbonate, polyurethane elastomer, acrylic, epoxy, or Delrin.

A11: The electrode cuff of any of A1 to A10, further comprising electronics that are used for one or more of sensing, signal conditioning, and stimulation of the nerve.

A12: The electrode cuff of any of A1 to A11, further comprising a sensor embedded in the first body or the top second body, wherein the sensor is configured to measure afferent traffic in a nerve within the device which occurs after and in response to stimulation by the electrodes.

A13: The electrode cuff of any of A1 to A12, further comprising a hinged joint between the first body and the second body, wherein the hinged joint is configured to accommodate nerve swelling.

A14: The electrode cuff of any of A1 to A13, further comprising a preformed lead formed of thermoformed encapsulation.

A15: The electrode cuff of A15, wherein the preformed lead includes a tension string.

A16: The electrode cuff of any of A1 to A15, wherein the bottom part includes spring-loaded hooks configured to secure the bottom part to muscle or other tissue.

A17: The electrode cuff of any of A1 to A16, wherein the electrodes are arranged in parallel and form an electrical field gradient having a maximum value within a nerve passage through the cuff.

A18: The electrode cuff of any of A1 to A17, wherein the electrodes are configured that a center electrode of the electrodes is electrically negative and outer electrodes of the electrodes are electrically positive to maximize the capture.

A19: The electrode cuff of any of A1 to A18, wherein the electrodes are arranged non-symmetrically on the second body.

A20: The electrode cuff of any of A1 to A19, wherein the second body has holes on opposite sides of the second body, wherein the holes are configured to receive sutures.

A21: The electrode cuff of any of A1 to A20, wherein the electrode(s) of the cuff are arranged to generate an electrical field, wherein a maximum positive peak of a calculated E-field gradient that is based on the electrical field corresponds to a central location of the cuff.

A22: The electrode cuff of any of A1 to A20, wherein the electrode(s) of the cuff are arranged to generate an electrical field, wherein a maximum positive peak of a calculated E-field gradient that is based on the electrical field corresponds to a center the cuff.

A23: Wherein the one or more electrodes of a phrenic nerve stimulation and sensing device are arranged to generate an electrical field, wherein a maximum positive peak of a calculated E-field gradient that is based on the electrical field corresponds to a center the cuff.

A24: Wherein the one or more electrodes of a phrenic nerve stimulation and sensing device are arranged to generate an electrical field, wherein a maximum positive peak of a calculated E-field gradient that is based on the electrical field is centrally located within the generated electrical field.

1. An electrode cuff for stimulating a phrenic nerve of a patient, the electrode cuff comprising:
a body including a bottom panel, an upper panel, and a connecting bridge joined to a first edge of the bottom panel and a first edge of the upper panel, wherein the upper panel is a cantilever extending from the connecting bridge and to a second edge of the upper panel opposite to the first edge,
wherein at least an inner surface of the bottom panel, the upper panel, and the connecting bridge define a passage, wherein the passage is configured to receive the phrenic nerve through a gap between the second edge of the upper panel and the bottom panel; and
electrodes in at least one of the inner surfaces of the bottom panel, the upper panel, or the connecting bridge, wherein the electrodes are configured to electrically stimulate a phrenic nerve that is within the passage.

2. The electrode cuff of claim 1, further comprising:
an additional body that is configured to engage the body and define, with the bottom panel, the upper panel, and the connecting bridge, the passage that is configured to receive the phrenic nerve of a patient.

3. The electrode cuff of claim 2, wherein the additional body is configured to engage a side of the bottom panel.

4. The electrode cuff of claim 2, wherein the body and the additional body are each rigid single piece components.

5. The electrode cuff of claim 2, wherein the body and the additional body form an oblate ellipsoid.

6. The electrode cuff of claim 2, further comprising a hinge connecting the body and the additional body.

7. The electrode cuff of claim 2, further comprising a sensor embedded in the body or the additional body, wherein the sensor is configured to measure afferent traffic in the phrenic nerve which occurs after and in response to stimulation by the electrodes.

8. The electrode cuff of claim 2, wherein the body and the additional body are both shaped to form an oblate ellipsoid when joined together, such that a side parallel to the major axis of a interior ellipsoidal space is flat, and a side perpendicular to the major axis of the interior ellipsoidal space is curved.

9. The electrode cuff of claim 1, further comprising: a hollow sleeve attached to the bottom panel configured to receive a conductive lead conductively attached to the electrodes.

10. The electrode cuff of claim 1, wherein at least one of the electrodes is conductively connected to another one of the electrodes.

11. The electrode cuff of claim 1, wherein an axis of a sleeve is parallel to a major axis that defines the passage in which the phrenic nerve is configured to be received.

12. The electrode cuff of claim 1, wherein the one or more electrodes are arranged to generate an electrical field, wherein a maximum positive peak of a calculated E-field gradient that is based on the electrical field corresponds to a center of a cuff.

13. The electrode cuff of claim 1, wherein the one or more electrodes are arranged to generate an electrical field, wherein a maximum positive peak of a calculated E-field gradient that is based on the electrical field is centrally located within the generated electrical field.

14. The electrode cuff of claim 1, further comprising electronics that are used for one or more of sensing, signal conditioning, and stimulation of the nerve.

15. The electrode cuff of claim 1, wherein the electrodes are arranged in parallel and form an electrical field gradient has a maximum value within a nerve passage through a cuff.

16. The electrode cuff of claim 1, wherein the electrodes are configured such that a center electrode of the electrodes is electrically negative and outer electrodes of the electrodes are electrically positive.

17. The electrode cuff of claim 1, wherein the electrodes are arranged non-symmetrically.

18. A phrenic nerve stimulation and sensing device according to claim 1 comprising:
the electrode cuff;
an in-situ suturing structure for fixation to muscle; and
a preformed lead conductively connected to electrodes in the body.

19. The phrenic nerve stimulation and sensing device of claim 18, wherein the preformed lead is formed of thermoformed encapsulation, or the preformed lead includes a tension string.

20. A phrenic nerve stimulation and sensing device comprising:
a first body with inner and outer surfaces in a form of an ellipsoidal dome;
a second body with a flat inner surface and a flat outer surface where the inner surface contains one or more electrodes;
electrodes that are located in the second body and oriented towards a passage formed by an assembly of the first body and the second body;
an in-situ suturing mechanism for fixation to muscle; and
a preformed lead conductively connected to electrodes in the second body.
